(19) United States
(12) Patent Application Publication
(10) Pub. No.: US 2004/0235867 A1
(43) Pub. Date: Nov. 25, 2004

(54) Tyrosine kinase inhibitors

(76) Inventors: Mark T. Bilodeau, Lansdale, PA (US); Peter J. Manley, Harleysville, PA (US); George D. Hartman, Lansdale, PA (US)

Correspondence Address: MERCK AND CO INC, P O BOX 2000, RAHWAY, NJ, 070650907

(21) Appl. No.: 10/484,986

(22) PCT Filed: Jul. 19, 2002
(86) PCT No.: PCT/US02/23191

Related U.S. Application Data
(60) Provisional application No. 60/307,443, filed on Jul. 24, 2001.

(51) Int. Cl. C07D 471/02; C07D 491/02; A61K 31/519; A61K 31/52

(52) U.S. Cl. 514/260.1; 514/263.2; 544/255; 544/277; 514/303; 514/301; 514/302; 546/114; 546/115; 546/117

(57) ABSTRACT

The present invention relates to compounds which inhibit, regulate and/or modulate tyrosine kinase signal transduction, compositions which contain these compounds, and methods of using them to treat tyrosine kinase-dependent diseases and conditions, such as angiogenesis, cancer, tumor growth, atherosclerosis, age related macular degeneration, diabetic retinopathy, inflammatory diseases, and the like in mammals.

### BACKGROUND OF THE INVENTION

The present invention relates to compounds which inhibit, regulate and/or modulate tyrosine kinase signal transduction, compositions which contain these compounds, and methods of using them to treat tyrosine kinase-dependent diseases and conditions, such as angiogenesis, cancer, tumor growth, atherosclerosis, age related macular degeneration, diabetic retinopathy, inflammatory diseases, and the like in mammals.

The following is provided as background information only and should not be taken as an admission that any subject matter discussed or that any reference mentioned is prior art to the instant invention.

Tyrosine kinases are a class of enzymes that catalyze the transfer of the terminal phosphate of adenosine triphosphate to tyrosine residues in protein substrates. Tyrosine kinases are believed, by way of substrate phosphorylation, to play critical roles in signal transduction for a number of cell functions. Though the exact mechanism of signal transduction is still unclear, tyrosine kinases have been shown to be important contributing factors in cell proliferation, carcinogenesis, cell differentiation and apoptosis.

Tyrosine kinases can be categorized as receptor type or non-receptor type. Receptor type tyrosine kinases have an extracellular, a transmembrane, and an intracellular portion, while non-receptor type tyrosine kinases are wholly intracellular.

The receptor type tyrosine kinases are comprised of a large number of transmembrane receptors with diverse biological activity. In fact, about twenty different subfamilies of receptor type tyrosine kinases have been identified. One tyrosine kinase subfamily, designated the HER subfamily, is comprised of EGFR, HER2, HER3, and HER4. Ligands of this subfamily of receptors include epithileal growth factor, TGF-α, amphiregulin, HB-EGF, betacellulin and heregulin. Another subfamily of these receptor type tyrosine kinases is the insulin subfamily, which includes INS-R, IGF-R, and IR-R The PDGF subfamily includes the PDGF-α and -β receptors, CSFIR, c-kit and FLK-II. Then there is the FLK family which is comprised of the kinase insert domain receptor (KDR), fetal liver kinase-1 (FLK-1), fetal liver kinase-4 (FLK-4) and the fms-like tyrosine kinase-1 (flt-1). The PDGF and FLK families are usually considered together due to the similarities of the two groups. For a detailed discussion of the receptor type tyrosine kinases, see Plowman et al., DN&P 7(6):334-339 (1994), which is hereby incorporated by reference.

The non-receptor type of tyrosine kinases is also comprised of numerous subfamilies, including Src, Frk, Btk, Csk, Abl, Zap70, Fes/Fps, Fak, Jak, Ack, and LIMK. Each of these subfamilies is further sub-divided into varying receptors. For example, the Src subfamily is one of the largest and includes Src, Yes, Fyn, Lyn, Lck, Blk, Hck, Fgr, and Yrk. The Src subfamily of enzymes has been linked to oncogenesis. For a more detailed discussion of the non-receptor type of tyrosine kinases, see Bolen, Oncogene 8:2025-2031 (1993), which is hereby incorporated by reference.

Both receptor type and non-receptor type tyrosine kinases are implicated in cellular signaling pathways leading to numerous pathogenic conditions, including cancer, psoriasis and hyperimmune responses.

Several receptor type tyrosine kinases, and the growth factors that bind thereto, have been suggested to play a role in angiogenesis, although some may promote angiogenesis indirectly. Mustonen and Alitalo, J. Cell Biol. 129:895-898 (1995). One such receptor type tyrosine kinase is fetal liver kinase 1 or FLK-1. The human analog of FLK-1 is the kinase insert domain-containing receptor KDR, which is also known as vascular endothelial cell growth factor receptor 2 or VEGFR-2, since it binds VEGF with high affinity. Finally, the murine version of this receptor has also been called NYK. Oelrichs et al., Oncogene 8(1):11-15 (1993). VEGF and KDR are a ligand-receptor pair that play an important role in the proliferation of vascular endothelial cells, and the formation and sprouting of blood vessels, termed vasculogenesis and angiogenesis, respectively.

Angiogenesis is characterized by excessive activity of vascular endothelial growth factor (VEGF). VEGF is actually comprised of a family of ligands. Klagsburn and D'Amore, Cytokine & Growth Factor Reviews 7:259-270 (1996). VEGF binds the high affinity membrane-spanning tyrosine kinase receptor KDR and the related fms-like tyrosine kinase-1, also known as Flt-1 or vascular endothelial cell growth factor receptor 1 (VEGFR-1). Cell culture and gene knockout experiments indicate that each receptor contributes to different aspects of angiogenesis. KDR mediates the mitogenic function of VEGF whereas Flt-1 appears to modulate non-mitogenic functions such as those associated with cellular adhesion. Inhibiting KDR thus modulates the level of mitogenic VEGF activity. In fact, tumor growth has been shown to be susceptible to the antiangiogenic effects of VEGF receptor antagonists. Kim et al., Nature 362:841-844 (1993).

Solid tumors can therefore be treated by tyrosine kinase inhibitors since these tumors depend on angiogenesis for the formation of the blood vessels necessary to support their growth. These solid tumors include histiocytic lymphoma, cancers of the brain, genitourinary tract, lymphatic system, stomach, larynx and lung, including lung adenocarcinoma and small cell lung cancer. Additional examples include cancers in which overexpression or activation of Raf-activating oncogenes (e.g., K-ras, erb-B) is observed. Such cancers include pancreatic and breast carcinoma. Accordingly, inhibitors of these tyrosine kinases are useful for the prevention and treatment of proliferative diseases dependent on these enzymes.

The angiogenic activity of VEGF is not limited to tumors. VEGF accounts for most of the angiogenic activity produced in or near the retina in diabetic retinopathy. This vascular growth in the retina leads to visual degeneration culminating in blindness. Ocular VEGF mRNA and protein are elevated by conditions such as retinal vein occlusion in primates and decreased pO₂ levels in mice that lead to neovascularization. Intraocular injections of anti-VEGF monoclonal antibodies or VEGF receptor immunofusions inhibit ocular neovascularization in both primate and rodent models. Regardless of the cause of induction of VEGF in human diabetic retinopathy, inhibition of ocular VEGF is useful in treating the disease.

Expression of VEGF is also significantly increased in hypoxic regions of animal and human tumors adjacent to areas of necrosis. VEGF is also upregulated by the expression of the oncogenes ras, raf, src and mutant p53 (all of which are relevant to targeting cancer). Monoclonal anti-VEGF antibodies inhibit the growth of human tumors in nude mice. Although these same tumor cells continue to express VEGF in culture, the antibodies do not diminish their mitotic rate. Thus tumor-derived VEGF does not function as an autocrine mitogenic factor. Therefore, VEGF contributes to tumor growth in vivo by promoting angiogenesis through its paracrine vascular endothelial cell chemotactic and mitogenic activities. These monoclonal antibodies also inhibit the growth of typically less well vascularized human colon cancers in athymic mice and decrease the number of tumors arising from inoculated cells.

Viral expression of VEGF-binding constructs of Flk-1 or Flt-1 (the mouse KDR receptor homologue), truncated to eliminate the cytoplasmic tyrosine kinase domains but retain the membrane anchors, virtually abolishes the growth of a transplantable glioblastoma in mice. Tumor growth is abolished presumably by a dominant negative mechanism during VEGF receptor heterodimerization. Embryonic stem cells, which normally grow as solid tumors in nude mice, do not produce detectable tumors if both VEGF alleles are knocked out. Taken together, these data indicate the role of VEGF in the growth of solid tumors. Inhibition of KDR or Flt-1 is implicated in pathological angiogenesis, and these receptors are useful in the treatment of diseases in which angiogenesis is part of the overall pathology, e.g., inflammation, diabetic retinal vascularization, as well as various forms of cancer since tumor growth is known to be dependent on angiogenesis. Weidner et al., N. Engl. J. Med. 324:1-8 (1991).

Accordingly, the identification of small compounds which specifically inhibit, regulate and/or modulate the signal transduction of tyrosine kinases is desirable and is an object of this invention.

### SUMMARY OF THE INVENTION

The present invention relates to compounds that are capable of inhibiting, modulating and/or regulating signal transduction of both receptor type and non-receptor type tyrosine kinases. One embodiment of the present invention is illustrated by a compound of Formula I, and the pharmaceutically acceptable salts and stereoisomers thereof:

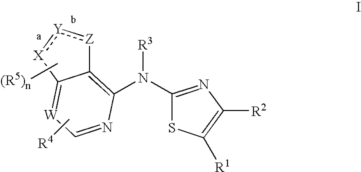

### DETAILED DESCRIPTION OF THE INVENTION

The compounds of the instant invention are useful in the inhibition of kinases and are illustrated by a compound of Formula I:

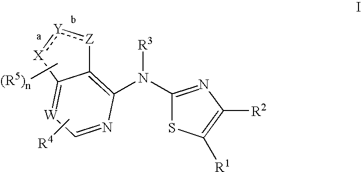

or a pharmaceutically acceptable salt or stereoisomer thereof, wherein

a and b are a single bond or a double bond provided both a and b are not a double bond at the same time;

X, Y and Z are C, S, N or O provided that at least one of X, Y and Z is C;

W is C or N;

n is 0 through 6;

R¹ is:

1) H,

2) Oᵣ(C₁-C₆)perfluoroalkyl,

3) OH,

4) CN,

5) halogen,

6) (C═O)ᵣOₛ(C₁-C₁₀)alkyl,

7) (C═O)ᵣOₛ(C₂-C₁₀)alkenyl,

8) (C═O)ᵣOₛ(C₂-C₁₀)alkynyl,

9) (C═O)ᵣOₛaryl,

10) (C═O)ᵣOₛheterocyclyl, or

11) (C₀-C₆)alkyl-NR^{a}R^{b},

wherein r and s are independently 0 or 1, and said alkyl, alkenyl, alkyl, aryl and heterocyclyl is optionally substituted with one or more substituents selected from R⁵;

R² is:

1) H,

2) Oᵣ(C₁-C₆)perfluoroalkyl,

3) OH,

4) CN,

5) halogen,

6) (C═O)ᵣOₛ(C₁-C₁₀)alkyl,

7) (C═O)ᵣOₛ(C₂-C₁₀)alkenyl,

8) (C═O)ᵣOₛ(C₂-C₁₀)alkynyl;

9) (C═O)ᵣOₛaryl,

10) (C═O)ᵣOₛheterocyclyl, or

11) (C₀-C₆)alkyl-NR^{a}R^{b},

wherein r and s are independently 0 or 1, and said alkyl, alkenyl, alkynyl, aryl and heterocyclyl is optionally substituted with one or more substituents selected from R⁵;

R³ is:

1) H,

2) SO₂R^{c},

3) (C═O)ᵣR^{c}, wherein r is 0 or 1, or

4) CO₂R^{c};

R⁴ is:

1) H,

2) Oᵣ(C₁-C₆)perfluoroalkyl,

3) OH,

4) CN,

5) halogen,

6) (C═O)ᵣOₛ(C₁-C₁₀)alkyl,

7) (C═O)ᵣOₛ(C₂-C₁₀)alkenyl,

8) (C═O)ᵣOₛ(C₂-C₁₀)alkynyl,

9) (C═O)ᵣOₛaryl,

10) (C═O)ᵣOₛheterocyclyl, or

11) (C₀-C₆)alkyl-NR^{a}R^{b},

wherein r and s are independently 0 or 1, and said alkyl, alkenyl, alkynyl, aryl and heterocyclyl is optionally substituted with one or more substituents selected from R⁵;

R⁵ is:

1) H,

2) SO₂R^{c},

3) (C═O)ᵣR^{c},

4) CO₂R^{c},

5) Oᵣ(C═O)ₛNR^{a}R^{b},

6) (C═O)ᵣOₛaryl,

7) (C═O)ᵣOₛ-heterocyclyl,

8) halogen,

9) OH,

10) oxo,

11) O(C₁-C₃)perfluoroalkyl,

12) (C₁-C₃)perfluoroalkyl,

13) (C═O)ᵣOₛ(C₁-C₁₀)alkyl,

14) CHO,

15) CO₂H, or

16) CN,

wherein r and s are independently 0 or 1, and said alkyl, aryl, and heterocyclyl are optionally substituted with one or more substituents selected from R^{d};

R^{a} and R^{b} are independently:

1) H,

2) (C═O)ᵣ(C₁-C₁₀)alkyl,

3) S(O)₂R^{c},

4) (C═O)ᵣheterocyclyl,

5) (C═O)ᵣaryl, or

6) CO₂R^{c},

wherein r is 0 or 1 and said alkyl, heterocyclyl, and aryl optionally substituted with one or more substituents selected from R^{d}, or

R^{a} and R^{b} are taken together with the nitrogen to which they are attached to form a monocyclic or bicyclic heterocycle with 5-7 members in each ring and optionally containing, in addition to the nitrogen, one or two additional heteroatoms selected from N, O and S, said monocyclic or bicyclic heterocycle optionally substituted with one or more substituents selected from R^{d};

R^{c} is (C₁-C₆)alkyl, aryl, benzyl, or heterocyclyl;

R^{d} is:

1) (C═O)ᵣOₛ(C₁-C₁₀)alkyl, wherein r and s are independently 0 or 1, optionally substituted with up to three substituents selected from OH, (C₁-C₆)alkoxy, halogen, CN, oxo, N(R^{e})₂ and S(O)₂R^{c},

2) (C═O)N(R^{e})₂,

3) Oᵣ(C₁-C₃)perfluoroalkyl,

4) (C₀-C₆)alkylene-S(O)ₘR^{c}, wherein m is 0, 1 or 2,

5) oxo,

6) OH,

7) halogen,

8) CN,

9) (C₀-C₆)alkylene-aryl, optionally substituted with up to three substituents selected from R^{e},

10) (C₀-C₆)alkylene-heterocyclyl, optionally substituted with up to three substituents selected from R^{e},

11) (C₀-C₆)alkylene-N(R^{e})₂,

12) C(O)R^{c},

13) CO₂R^{c},

14) C(O)H, or

15) CO₂H; and

R^{e} is H, (C₁-C₆)alkyl, aryl, heterocyclyl or S(O)R^{c}.

A further embodiment is illustrated by a compound as described directly above of Formula I, wherein W is C or N; R¹ is CN or phenyl; and R², R³ and R⁴ are H.

Another embodiment is illustrated by a compound as described directly above of Formula I, wherein W is C and R¹ is CN.

A preferred embodiment is a compound selected from:

2-(2,3-dihydrofuro[2,3-c]pyridin-7-ylamino)-1,3-thiazole-5-carbonitrile;

2-{[3-(hydroxymethyl)-2,3-dihydrofuro[2,3-c]pyridin-7-yl]amino}-1,3-thiazole-5-carbonitrile;

2-[(1-methyl-1H-pyrazolo[4,3-c]pyridin-4-yl)amino]-1,3-thiazole-5-carbonitrile;

2-(2,3-dihydro-1H-pyrrolo[3,2-c]pyridin-4-ylamino)-1,3-thiazole-5-carbonitrile;

2-(1H-pyrrolo[3,2-c]pyridin-4-ylamino)-1,3-thiazole-5-carbonitrile;

2-{[1-(methylsulfonyl)-2,3-dihydro-1H-pyrrolo[3,2-c]pyridin-4-yl]amino}-1,3-thiazole-5-carbonitrile;

4-[(5-cyano-1,3-thiazol-2-yl)amino]-N,N-dimethyl-2,3-dihydro-1H-pyrrolo[3,2-c]pyridine-1-carboxamide;

2-[(1-methyl-2-oxo-2,3-dihydro-1H-imidazo[4,5-c]pyridin-4-yl)amino]-1,3-thiazole-5-carbonitrile;

2-(thieno[3,2-c]pyridin-4-ylamino)-1,3-thiazole-5-carbonitrile;

2-(furo[3,2-c]pyridin-4-ylamino)-1,3-thiazole-5-carbonitrile;

2-(thieno[2,3-d]pyrimidin-4-ylamino)-1,3-thiazole-5-carbonitrile;

2-{4-[(5-cyano-1,3-thiazol-2-yl)amino]-1H-pyrrolo[3,2-c]pyridin-1-yl}-N,N-diethylacetamide;

2-{4-[(5-Cyano-1,3-thiazol-2-yl)amino]-1H-pyrrolo[3,2-c]pyridin-1-yl)}-N,N-dimethylacetamide;

2-{[1-(2-oxo-2-piperazin-1-ylethyl)-1H-pyrrolo[3,2-c]pyridin-4-yl]amino)-1,3-thiazole-5-carbonitrile;

2-{3-Chloro-4-[(5-cyano-1,3-thiazol-2-yl)amino]-1H-pyrrolo[3,2-c]pyridin-1-yl}-N,N-dimethylacetamide;

2-{4-[(5-cyano-1,3-thiazol-2-yl)amino]-7H-pyrrolo[2,3-d]pyrimidin-7-yl}-N,N-diethylacetamide; and

2-{4-[(5-cyano-1,3-thiazol-2-yl)amino]-5,6-dihydro-7H-pyrrolo[2,3-d]pyrimidin-7-yl}-N,N-dimethylacetamide;

or a pharmaceutically acceptable salt or stereoisomer thereof.

Also included within the scope of the present invention is a pharmaceutical composition which is comprised of a compound of Formula I as described above and a pharmaceutically acceptable carrier. These and other aspects of the invention will be apparent from the teachings contained herein.

Utilities

The instant compounds are useful as pharmaceutical agents for mammals, especially for humans, in the treatment of tyrosine kinase dependent diseases. Such diseases include the proliferation of tumor cells, the pathologic neovascularization (or angiogenesis) that supports solid tumor growth, ocular neovascularization (diabetic retinopathy, age-related macular degeneration, and the like) and inflammation (psoriasis, rheumatoid arthritis, and the like).

The compounds of the instant invention may be administered to patients for use in the treatment of cancer. The instant compounds inhibit tumor angiogenesis, thereby affecting the growth of tumors. Rak et al., Cancer Research 55:4575-4580 (1995). The anti-angiogenesis properties of the instant compounds are also useful in the treatment of certain forms of blindness related to retinal vascularization.

The disclosed compounds are also useful in the treatment of certain bone-related pathologies, such as osteosarcoma, osteoarthritis, and rickets, also known as oncogenic osteomalacia. Hasegawa et al., Skeletal Radiol. 28:41-45 (1999); Gerber et al., Nature Medicine 5(6):623-628 (1999). And since VEGF directly promotes osteoclastic bone resorption through KDR/Flk-1 expressed in mature osteoclasts, FEBS Let. 473:161-164 (2000); Endocrinology 141:1667 (2000), the instant compounds are also useful to treat and prevent conditions related to bone resorption, such as osteoporosis and Paget's disease.

The claimed compounds can also be used to reduce or prevent tissue damage which occurs after cerebral ischemic events, such as stroke, by reducing cerebral edema, tissue damage, and reperfusion injury following ischemia. Drug News Perspect. 11:265-270 (1998); J. Clin. Invest. 104:1613-1620 (1999).

The instant compounds are useful in the treatment of preeclampsia. Studies have shown that the action of VEGF on the Flt-1 receptor is pivotal in the pathogenesis of preeclampsia. Laboratory Investigation 79:1101-1111 (1999). Vessels of pregnant women incubated with VEGF exhibit a reduction in endothelium-dependent relaxation similar to that induced by plasma from women with preeclampsia. In the presence of an anti-Flt-1 receptor antibody, however, neither VEGF or plasma from women with preeclampsia reduced the endothelium-dependent relaxation. Therefore the claimed compounds serve to treat preeclampsia via their action on the tyrosine kinase domain of the Flt-1 receptor.

The instant compounds can also be used to prevent or treat tissue damage during bacterial meningitis. (Matsuyama et al., J. Neurol. Sci. 186:75-79 (2001)). The instant invention therefore encompasses a method of treating or preventing tissue damage due to bacterial meningitis which comprises administering a therapeutically effective amount of a compound of Formula I. Studies have shown that VEGF is secreted by inflammatory cells during bacterial meningitis and that VEGF contributes to blood-brain barrier disruption. (van der Flier et al., J. Infectious Diseases, 183:149-153 (2001)). The claimed compounds can inhibit VEGF-induced vascular permeability and therefore serve to prevent or treat blood-brain barrier disruption associated with bacterial meningitis.

The compounds of this invention may be administered to mammals, preferably humans, either alone or, preferably, in combination with pharmaceutically acceptable carriers or diluents, optionally with known adjuvants, such as alum, in a pharmaceutical composition, according to standard pharmaceutical practice. The compounds can be administered orally or parenterally, including the intravenous, intramuscular, intraperitoneal, subcutaneous, rectal and topical routes of administration.

For oral use of a chemotherapeutic compound according to this invention, the selected compound may be administered, for example, in the form of tablets or capsules, or as an aqueous solution or suspension. In the case of tablets for oral use, carriers which are commonly used include lactose and corn starch, and lubricating agents, such as magnesium stearate, are commonly added. For oral administration in capsule form, useful diluents include lactose and dried corn starch. When aqueous suspensions are required for oral use, the active ingredient is combined with emulsifying and suspending agents. If desired, certain sweetening and/or flavoring agents may be added. For intramuscular, intraperitoneal, subcutaneous and intravenous use, sterile solutions of the active ingredient are usually prepared, and the pH of the solutions should be suitably adjusted and buffered. For intravenous use, the total concentration of solutes should be controlled in order to render the preparation isotonic.

The compounds of the instant invention may also be co-administered with other well known therapeutic agents that are selected for their particular usefulness against the condition that is being treated. For example, in the case of bone-related disorders, combinations that would be useful include those with antiresorptive bisphosphonates, such as alendronate and risedronate; integrin blockers (defined further below), such as αᵥβ₃ antagonists; conjugated estrogens used in hormone replacement therapy, such as PREMPRO®, PREMARIN® and ENDOMBTRION®; selective estrogen receptor modulators (SERMs), such as raloxifene, droloxifene, CP-336,156 (Pfizer) and lasofoxifene; cathespin K inhibitors; and ATP proton pump inhibitors.

The instant compounds are also useful in combination with known anti-cancer agents. Such known anti-cancer agents include the following: estrogen receptor modulators, androgen receptor modulators, retinoid receptor modulators, cytotoxic agents, antiproliferative agents, prenyl-protein transferase inhibitors, IMG-CoA reductase inhibitors, HIV protease inhibitors, reverse transcriptase inhibitors, and other angiogenesis inhibitors. The instant compounds are particularly useful when co-administered with radiation therapy. The synergistic effects of inhibiting VEGF in combination with radiation therapy have been described in the art. (see WO 00/61186.)

“Estrogen receptor modulators” refers to compounds which interfere or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenylhydrazone, and SH646.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl) retinamide, N-4-carboxyphenyl retinamide,

“Cytotoxic agents” refer to compounds which cause cell death primarily by interfering directly with the cell's functioning or inhibit or interfere with cell myosis, including alkylating agents, tumor necrosis factors, intercalators, microtubulin inhibitors, and topoisomerase inhibitors.

Examples of cytotoxic agents include, but are not limited to, tirapazimine, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum(II)]tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycarminomycin, annamycin, galarubicin, elinafide, MEN10755, and 4-demethoxy-3-deamino-3-aziridinyl-4-methylsulphonyl-daunorubicin (see WO 00/50032).

Examples of microtubulin inhibitors include paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide, TDX258, and BMS188797.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H)propanamine, 1-amino-9-ethyl-5-fluoro-2,3-dihydro-9-hydroxy-4-methyl-1H,12H-benzo[de]pyrano[3′,4′:b,7]indolizino[1,2b]quinoline-10,13(9H,15H)dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′ dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a,5aB,8aa,9b)-9-[2-[N-[2-(dimethylamino)ethyl]-N-methylamino]ethyl]-5-[4-hydroxy-3,5-dimethoxyphenyl]-5,5a,6,8,8a,9-hexohydrofuro(3′,4′:6,7) naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo [g]isoguinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2 (diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one, and dimesna.

“Antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-flurouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N-4-palmitoyl-1-B-D-arabino furanosyl cytosine, and 3-aminopyridine-2-carboxaldehyde thiosemicarbazone. “Antiproliferative agents” also includes monoclonal antibodies to growth factors, other than those listed under “angiogenesis inhibitors”, such as trastuzumab, and tumor suppressor genes, such as p53, which can be delivered via recombinant virus-mediated gene transfer (see U.S. Pat. No. 6,069,134, for example).

“HMG-CoA reductase inhibitors” refers to inhibitors of 3-hydroxy-3-methylglutaryl-CoA reductase. Compounds which have inhibitory activity for HMG-COA reductase can be readily identified by using assays well-known in the art. For example, see the assays described or cited in U.S. Pat. No. 4,231,938 at col. 6, and WO 84/02131 at pp. 30-33. The terms “HMG-CoA reductase inhibitor” and “inhibitor of HMG-CoA reductase” have the same meaning when used herein.

Examples of HMG-CoA reductase inhibitors that may be used include but are not limited to lovastatin (EVACOR®; see U.S. Pat. Nos. 4,231,938, 4,294,926 and 4,319,039), simvastatin (ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896), atorvastatin (LIPITOR®; see U.S. Pat. Nos. 5,273,995, 4,681,893, 5,489,691 and 5,342,952) and cerivastatin (also known as rivastatin and BAYCHOL®; see U.S. Pat. No. 5,177,080). The structural formulas of these and additional HMG-COA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefor the use of such salts, esters, open-acid and lactone forms is included within the scope of this invention. An illustration of the lactone portion and its corresponding open-acid form is shown below as structures I and II.

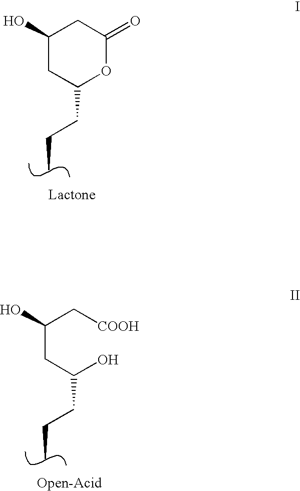

In HMG-COA reductase inhibitors where an open-acid form can exist, salt and ester forms may preferably be formed from the open-acid, and all such forms are included within the meaning of the term “HMG-CoA reductase inhibitor” as used herein. Preferably, the HMG-CoA reductase inhibitor is selected from lovastatin and simvastatin, and most preferably simvastatin. Herein, the term “pharmaceutically acceptable salts” with respect to the HMG-CoA reductase inhibitor shall mean non-toxic salts of the compounds employed in this invention which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, ornithine, choline, N,N′-dibenzylethylenediamine, chloroprocaine, diethanolamine, procaine, N-benzylphenethylamine, 1-p-chlorobenzyl-2-pyrrolidine-1′-yl-methyl-benzimidazole, diethylamine, piperazine, and tris(hydroxymethyl)aminomethane. Further examples of salt forms of HMG-CoA reductase inhibitors may include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylsulfate, mucate, napsylate, nitrate, oleate, oxalate, pamaote, palmitate, panthothenate, phosphate/diphosphate, polygalacturonate, salicylate, stearate, subacetate, succinate, tannate, tartrate, teoclate, tosylate, triethiodide, and valerate.

Ester derivatives of the described HMG-CoA reductase inhibitor compounds may act as prodrugs which, when absorbed into the bloodstream of a warm-blooded animal, may cleave in such a manner as to release the drug form and permit the drug to afford improved therapeutic efficacy.

“Prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase). Examples of prenyl-protein transferase inhibiting compounds include (±)-6-[amino(4-chlorophenyl)(1-methyl-1H-imidazol-5-yl)methyl]-4-(3-chlorophenyl)-1-methyl-2-(1H)-quinolinone, (−)-6-[amino(4-chlorophenyl)(1-methyl-1H-imidazol-5-yl)methyl]-4-(3-chlorophenyl)-1-methyl-2(1H)-quinolinone, (+)-6-[amino(4-chlorophenyl)(1-methyl-1H-imidazol-5-yl)methyl]-4-(3-chlorophenyl)-1-methyl-2(1H)-quinolinone, 5(S)-n-butyl-1-(2,3-dimethylphenyl)4[1-(4-cyanobenzyl)-5-imidazolylmethyl]-2-piperazinone, (S)-1-(3-chlorophenyl)-4-[1-(4-cyanobenzyl)-5-imidazolylmethyl]-5-[2-(ethanesulfonyl)methyl)-2-piperazinone, 5(S)-n-Butyl-1-(2-methylphenyl)4-[1-(4-cyanobenzyl)-5-imidazolylmethyl]-2-piperazinone, 1-(3-chlorophenyl) 1[1-(4-cyanobenzyl)-2-methyl-5-imidazolylmethyl]-2-piperazinone, 1-(2,2-diphenylethyl)-3-[N-(1-(4-cyanobenzyl)-1H-imidazol-5-ylethyl)carbamoyl]piperidine, 415-[4-Hydroxymethyl-4-(4-chloropyridin-2-ylmethyl)-piperidine-1-ylmethyl]-2-methylimidazol-1-ylmethyl}benzonitrile, 4-{5-[4-hydroxymethyl-4-(3-chlorobenzyl)-piperidine-1-ylmethyl]-2-methylimidazol-1-ylmethyl}benzonitrile, 4-{3-{[(2-oxo-2H-pyridin-1-yl)benzyl]-3H-imidazol-4-ylmethyl}benzonitrile, 4-{3-[4-(5-chloro-2-oxo-2H-[1,2′]bipyridin-5′-ylmethyl]-3H-imidazol-4-ylmethyl}benzonitrile, 4-{3-[4-(2-Oxo-2H-[1,2′]bipyridin-5′-ylmethyl]-3H-imidazol-4-ylmethyl}benzonitrile, 4-[3-(2-Oxo-1-phenyl-1,2-dihydropyridin-4-methyl)-3H-imidazol-4-ylmethyl}benzonitrile, 18,19-dihydro-19-oxo-5H,17H-6,10:12,16-dimetheno-1H-imidazo[4,3-c][1,11,4]dioxaazacyclo-nonadecine-9-carbonitrile, (±)-19,20-Dihydro-19-oxo-5H-18,21-ethano-12,14-etheno-6,10-metheno-22H-benzo[d]imidazo[4,3-k][1,6,9,12]oxatriaza-cyclooctadecine-9-carbonitrile, 19,20-dihydro-19-oxo-5H,17H-18,21-ethano-6,10:12,16-dimetheno-22H-imidazo[3,4-h][1,8,11,14]oxatriazacycloeicosine-9-carbonitrile, and (±)-19,20-Dihydro-3-methyl-19-oxo-5H-18,21-ethano-12,14-etheno-6,10-metheno-22H-benzo[dimidazo[4,3-k][1,6,9,12]oxa-triazacyclooctadecine-9-carbonitrile.

Other examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. No. 5,420,245, U.S. Pat. No. 5,523,430, U.S. Pat. No. 5,532,359, U.S. Pat. No. 5,510,510, U.S. Pat. No. 5,589,485, U.S. Pat. No. 5,602,098, European Patent Publ. 0 618 221, European Patent Publ. 0 675 112, European Patent Publ. 0 604 181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO 97/30053, WO 97/44350, WO 98/02436, and U.S. Pat. No. 5,532,359. For an example of the role of a prenyl-protein transferase inhibitor on angiogenesis see European J. of Cancer 35(9):1394-1401 (1999).

Examples of HIV protease inhibitors include amprenavir, abacavir, CGP-73547, CGP-61755, DMP-450, indinavir, nelfinavir, tipranavir, ritonavir, saquinavir, ABT-378, AG 1776, and BMS-232,632. Examples of reverse transcriptase inhibitors include delaviridine, efavirenz, GS-840, HB Y097, lamivudine, nevirapine, AZT, 3TC, ddC, and ddI.

“Angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR20), inhibitors of epidermal-derived, fibroblast-derived, or platelet derived growth factors, MMP (matrix metalloprotease) inhibitors, integrin blockers, interferon-α, interleukin-12, pentosan polysulfate, cyclooxygenase inhibitors, including nonsteroidal anti-inflammatories (NSAIDs) like aspirin and ibuprofen as well as selective cyclooxygenase-2 inhibitors like celecoxib and rofecoxib. (PNAS 89:7384 (1992); JNCI 69:475 (1982); Arch. Opthamol. 108:573 (1990); Anat. Rec. 238:68 (1994); FEBS Letters 372:83 (1995); Clin. Orthop. 313:76 (1995); J. Mol. Endocrinol. 16:107 (1996); Jpn. J. Pharmacol. 75:105 (1997); Cancer Res. 57:1625 (1997); Author, Cell 93:705 (1998); Intl. J. Mol. Med. 2:715 (1998); J. Biol. Chem. 274:9116 (1999)), carboxyamidotriazole, combretastatin A4, squalamine, 6-O(-chloroacetyl-carbonyl)-fumagillol, thalidomide, angiostatin, troponin-1, angiotensin II antagonists (see Fernandez et al., J. Lab. Clin. Med. 105:141-145 (1985)), and antibodies to VEGF. (see, Nature Biotechnology 17:963-968 (1999); Kim et al., Nature, 362:841-844 (1993); WO 00/44777; and WO 00/61186).

As described above, the combinations with NSAID's are directed to the use of NSAID's which are potent COX-2 inhibiting agents. For purposes of this specification an NSAID is potent if it possess an IC₅₀ for the inhibition of COX-2 of 1 μM or less as measured by the cell or microsomal assay disclosed herein.

The invention also encompasses combinations with NSAID's which are selective COX-2 inhibitors. For purposes of this specification NSAID's which are selective inhibitors of COX-2 are defined as those which possess a specificity for inhibiting COX-2 over COX-1 of at least 100 fold as measured by the ratio of IC₅₀ for COX-2 over IC₅₀ for COX-1 evaluated by the cell or micromsal assay disclosed hereinunder. Such compounds include, but are not limited to those disclosed in U.S. Pat. No. 5,474,995, issued Dec. 12, 1995, U.S. Pat. No. 5,861,419, issued Jan. 19, 1999, U.S. Pat. No. 6,001,843, issued Dec. 14, 1999, U.S. Pat. No. 6,020,343, issued Feb. 1, 2000, U.S. Pat. No. 5,409,944, issued Apr. 25, 1995, U.S. Pat. No. 5,436,265, issued Jul. 25, 1995, U.S. Pat. No. 5,536,752, issued Jul. 16, 1996, U.S. Pat. No. 5,550,142, issued Aug. 27, 1996, U.S. Pat. No. 5,604,260, issued Feb. 18, 1997, U.S. Pat. No. 5,698,584, issued Dec. 16, 1997, U.S. Pat. No. 5,710,140, issued Jan. 20, 1998, WO 94/15932, published Jul. 21, 1994, U.S. Pat. No. 5,344,991, issued Jun. 6, 1994, U.S. Pat. No. 5,134,142, issued Jul. 28, 1992, U.S. Pat. No. 5,380,738, issued Jan. 10, 1995, U.S. Pat. No. 5,393,790, issued Feb. 20, 1995, U.S. Pat. No. 5,466,823, issued Nov. 14, 1995, U.S. Pat. No. 5,633,272, issued May 27, 1997, and U.S. Pat. No. 5,932,598, issued Aug. 3, 1999, all of which are hereby incorporated by reference.

Other examples of specific inhibitors of COX-2 include the following:

3-(3-fluorophenyl)-4-(4-(methylsulfonyl)phenyl)-2-(5H)-furanone;

3-(3,4-difluorophenyl)4-(4-(methylsulfonyl)phenyl)-2-(5H)-furanone;

3-(3,4-dichlorophenyl)4-(4-(methylsulfonyl)phenyl)-2-(5H)-furanone;

3-phenyl-4-(4-(methylsulfonyl)phenyl)-2-(5H)-furanone;

5,5-dimethyl-3-(3-fluorophenyl)4-(methylsulfonyl)phenyl)-2-(5H)-furanone;

3-(4-methylsulfonyl)phenyl-2-phenyl-5-trifluoromethylpyridine;

2-(3-chlorophenyl)-3-(4-methylsulfonyl)phenyl-5-trifluoromethyl-pyridine;

2-(4-chlorophenyl)-3-(4-methylsulfonyl)phenyl-5-trifluoromethyl-pyridine;

2-(4-fluorophenyl)-3-(4-methylsulfonyl)phenyl-5-trifluoromethyl-pyridine;

3-(4-methylsulfonyl)phenyl-2-(3-pyridinyl)-5-trifluoromethylpyridine;

5-methyl-3-(4-methylsulfonyl)phenyl-2-phenylpyridine;

2-(4-chlorophenyl)-5-methyl-3-(4-methylsulfonyl)phenylpyridine;

5-methyl-3-(4-methylsulfonyl)phenyl-2-(3-pyridinyl)pyridine;

5-chloro-2-(4-chlorophenyl)-3-(4-methylsulfonyl)phenylpyridine;

5-chloro-3-(4-methylsulfonyl)phenyl-2-(2-pyridinyl)pyridine;

5-chloro-3-(4-methylsulfonyl)phenyl-2-(3-pyridinyl)pyridine;

5-chloro-3-(4-methylsulfonyl)phenyl-2-(4-pyridinyl)pyridine;

5-chloro-3-(4-methylsulfonyl)phenyl-2-(2-methyl-5-pyridinyl)pyridine;

2-(4-chlorophenyl)-3-(4-methylsulfonyl)phenylpyridinyl-5-carboxylic acid methyl ester;

2-(4-chlorophenyl)-3-(4-methylsulfonyl)phenylpyridinyl-5-carboxylic acid;

5-cyano-2-(4-chlorophenyl)-3-(4-methylsulfonyl)phenylpyridine;

5-chloro-3-(4-methylsulfonyl)phenyl-2-(3-pyridyl)pyridine hydromethanesulfonate;

5-chloro-3-(4-methylsulfonyl)phenyl-2-(3-pyridyl)pyridine hydrochloride;

5-chloro-3-(4-methylsulfonyl)phenyl-2-(2-methyl-5-pyridinyl)pyridine hydrochloride;

5-chloro-3-(4-methylsulfonyl)phenyl-2-(2-ethyl-5-pyridinyl)pyridine;

5-chloro-3-(4-methylsulfonyl)phenyl-2-(2-ethyl-5-pyridinyl)pyridine hydromethanesulfonate;

3-(3,4-difluorophenoxy)-5,5-dimethyl-4-(4-(methylsulfonyl)phenyl)-5H-furan-2-one;

3-(3-fluorophenoxy)-5,5-dimethyl(4-(methylsulfonyl)phenyl)-5H-furan-2-one;

3-(3,5-difluorophenoxy)-5,5-dimethyl-4-(methylsulfonyl)phenyl)-5H-furan-2-one;

3-phenoxy-5,5-dimethyl-4-(4-(methylsulfonyl)phenyl)-5H-furan-2-one;

3-(2,4-difluorophenoxy)-5,5-dimethyl-4-(4-(methylsulfonyl)phenyl)-5H-furan-2-one;

3-(4-chlorophenoxy)-5,5-dimethyl-4-(4-(methylsulfonyl)phenyl)-5H-furan-2-one;

3-(3,4-dichlorophenoxy)-5,5-dimethyl-4-(methylsulfonyl)phenyl)-5H-furan-2-one;

3-(4-fluorophenoxy)-5,5-dimethyl-4-(4-(methylsulfonyl)phenyl)-5H-furan-2-one;

3-(4-fluorophenylthio)-5,5-dimethyl-4-(4-(methylsulfonyl)phenyl)-5H-furan-2-one;

3-(3,5-difluorophenylthio)-5,5-dimethyl-4-(4-(methylsulfonyl)phenyl)-5H-furan-2-one;

3-phenylthio-5,5-dimethyl-4-(4-(methylsulfonyl)phenyl)-5H-furan-2-one;

3-(N-phenylamino)-5,5-dimethyl(4-(methylsulfonyl)phenyl)-5H-furan-2-one;

3-(N-methyl-N-phenylamino)-5,5-dimethyl-4-(4-(methylsulfonyl)phenyl)-5H-furan-2-one;

3-cyclohexyloxy-5,5-dimethyl-4-(4-(methylsulfonyl)phenyl)-5H-furan-2-one;

3-phenylthio-4-(4-(methylsulfonyl)phenyl)-5H-furan-2-one;

3-benzyl-5,5-dimethyl-4-(4-(methylsulfonyl)phenyl)-5H-furan-2-one;

3-(3,4-difluorophenylhydroxymethyl)-5,5-dimethyl-4-(4-(methylsulfonyl)phenyl)-5H-furan-2-one;

3-(3,4-difluorobenzoyl)-5,5-dimethyl-4-(4-(methylsulfonyl)phenyl)-5H-furan-2-one;

3-benzoyl-5,5-dimethyl-4-(4-(methylsulfonyl)phenyl)-5H-furan-2-one;

4-(4-(methylsulfonyl)phenyl)-3-phenoxy-1-oxaspiro[4.4]non-3-en-2-one;

4-(4-(methylsulfonyl)phenyl)-3-phenylthio-1-oxaspiro[4.4]non-3-en-2-one;

4-(2-oxo-3-phenylthio-1-oxa-spiro[4,4]non-3-en-4-yl)benzenesulfonamide;

3-(4-fluorobenzyl)-5,5-dimethyl-4-(4-(methylsulfonyl)phenyl)-5H-furan-2-one;

3-(3,4-difluorophenoxy)-5-methoxy-5-methyl-4-(4-(methylsulfonyl)phenyl)-5H-furan-2-one;

3-(5-chloro-2-pyridyloxy)-5,5-dimethyl-4-(4-(methylsulfonyl)phenyl)-5H-furan-2-one;

3-(2-pyridyloxy)-5,5-dimethyl-4-(4-(methylsulfonyl)phenyl)-5H-furan-2-one;

3-(6-methyl-2-pyridyloxy)-5,5-dimethyl-4-(4-(methylsulfonyl)phenyl)-5H-furan-2-one;

3-(3-isoquinolinoxy)-5,5-dimethyl-4-(4-(methylsulfonyl)phenyl)-5H-furan-2-one;

3-(4-(methylsulfonyl)phenyl)-2-phenoxycyclopent-2-enone;

3-(4-(methylsulfonyl)phenyl)-2-(3,4-difluorophenoxy)cyclopent-2-enone;

5,5-dimethyl(4-methylsulfonylphenyl)-3-(5-bromopyridin-2-yloxy)-5H-furan-2-one;

5,5-dimethyl-4-(4-methylsulfonylphenyl)-3-(2-propoxy)-5H-furan-2-one;

2-(3,4-difluorophenoxy)-3-(4-methylsulfonylphenyl)-cyclopent-2-enone;

3-(5-benzothiophenyloxy)-5,5-dimethyl-4-(4-(methylsulfonyl)phenyl)-5H-furan-2-one;

5,5-dimethyl-4-(4-methylsulfonyl-phenyl)-3-(pyridyl-4-oxy)-5H-furan-2-one;

5,5-dimethyl-4-(4-methylsulfonyl-phenyl)-3-(pyridyl-3-oxy)-5H-furan-2-one;

3-(2-methyl-5-pyridyloxy)-5,5-dimethyl-4-(4-(methylsulfonyl)phenyl)-5H-furan-2-one;

3-(2-fluoro-4-trifluoromethyl)phenoxy-4-(4-methylsulfonyl)phenyl)-5,5-dimethyl-5H-furan-2-one;

3-(5-chloro-2-pyridylthio)-5,5-dimethyl-4-(4-methylsulfonyl)phenyl-5H-furan-2-one;

2-(3,5-difluorophenoxy)-3-(4-methylsulfonylphenyl)-cyclopent-2-enone;

3-(2-pyrimidinoxy)-5,5-dimethyl-4-(4-methylsulfonyl)phenyl-5H-furan-2-one;

3-(3-methyl-2-pyridyloxy)-5,5-dimethyl-4-(4-methylsulfonyl)phenyl-5H-furan-2-one;

3-(3-chloro-5-pyridyloxy)-5,5-dimethyl-4-(4-(methylsulfonyl)phenyl)-5H-furan-2-one;

3-(3-(1,2,5-thiadiazolyl)oxy)-4-(4-(methylsulfonyl)phenyl)-5,5-dimethyl-5H-furan-2-one;

3-(5-isoquinolinoxy)-5,5-dimethyl-4-(4-methylsulfonyl)phenyl-5H-furan-2-one;

3-(6-amino-2-pyridyloxy)-5,5-dimethyl-4-(4-(methylsulfonyl)phenyl)-5H-furan-2-one;

3-(3-chloro-4-fluoro)phenoxy-4-(methylsulfonyl)phenyl)-5;5-dimethyl-5H-furan-2-one;

3-(6-quinolinoxy)-5,5-dimethyl-4-(4-(methylsulfonyl)phenyl)-5H-furan-2-one;

3-(5-nitro-2-pyridyloxy)-5,5-dimethyl-4-(4-methylsulfonyl)phenyl-5H-furan-2-one;

3-(2-thiazolylthio)-5,5-dimethyl-4-(4-(methylsulfonyl)phenyl)-5H-furan-2-one;

3-(3-chloro-5-pyridyloxy)-5,5-dimethyl-4-(4-(methylsulfonyl)phenyl)-5H-furan-2-one;

5,5-dimethyl-4-(4-methylsulfonylphenyl)-3-(2-propoxy)-5H-furan-2-one;

3-(3-trifluoromethyl)phenoxy-4-(4-methylsulfonyl)phenyl)-5,5-dimethyl-5H-furan-2-one;

5,5-dimethyl-(4-(4-methylsulfonyl)phenyl)-3-(piperidine-1-carbonyl)-5-H-furan-2-one;

5,5-dimethyl-3-(2-butoxy)4-(4-methylsulfonylphenyl)-5H-furan-2-one;

5,5-dimethyl-4-(4-methylsulfonylphenyl)-3-(3-pentoxy)-5H-furan-2-one;

2-(5-chloro-2-pyridyloxy)-3-(4-methylsulfonyl)phenylcyclopent-2-enone;

3-(4-methyl-2-pyridyloxy)-5,5-dimethyl-4-(4-methylsulfonyl)phenyl-5H-furan-2-one;

(5R)-3-(3,4-difluorophenoxy)-5-ethyl-5-methyl(4-methylsulfonyl)phenyl-5H-furan-2-one;

(5R)-3-(4-chlorophenoxy)-5-ethyl-5-methyl-4-(4-methylsulfonyl)phenyl-5H-furan-2-one;

3-(2-methyl-3-pyridyloxy)-5,5-dimethyl-4-(4-methylsulfonyl)phenyl-5H-furan-2-one;

3-(4-methyl-5-nitro-2-pyridyloxy)-5,5-dimethyl-4-(4-methylsulfonyl)phenyl-5H-furan-2-one;

3-(5-chloro-4-methyl-2-pyridyloxy)-5,5-dimethyl-4-(4-methylsulfonyl)phenyl-5H-furan-2-one,

3-(5-fluoro-4-methyl-2-pyridyloxy)-5,5-dimethyl-4-(4-methylsulfonyl)phenyl-5H-furan-2-one;

3-(3-chloro-2-pyridyloxy)-5,5-dimethyl-4-(4-methylsulfonyl)phenyl-5H-furan-2-one;

3-(4-fluorophenoxy)-5-methyl-4-(4-methylsulfonyl)phenyl-5-propyl-5H-furan-2-one;

3-(N,N-diethylamino)-5,5-dimethyl-4-(4-(methylsulfonyl)phenyl)-5H-furan-2-one;

5,5-dimethyl-4-(4-methylsulfonyl-phenyl)-3-(3,5-dichloro-2-pyridyloxy)-5H-furan-2-one;

(5R)-3-(4-bromophenoxy)-5-ethyl-5-methyl-4-(4-methylsulfonyl)phenyl-5H-furan-2-one;

(5R)-3-(4-methoxyphenoxy)-5-ethyl-5-methyl-4-(4-methylsulfonyl)phenyl-5H-furan-2-one;

(5R)-3-(5-chloro-2-pyridyloxy)-5-methyl-4-(4-methylsulfonyl)phenyl-5-(2,2,2-trifluoroethyl)-5H-furan-2-one;

3-(5-chloro-2-pyridyloxy)-5-methyl-4-(4-methylsulfonyl)phenyl-5-propyl-5H-furan-2-one;

3-(1-cyclopropyl-ethoxy)-5,5 diethyl-4-(4-methyl sulfonyl)phenyl)-5H-furan-2-one;

5-methyl-4-(4-(methylsulfonyl)phenyl)-3-(2-(propoxy)-5-(2-trifluoroethyl)-5H-furan-2-one;

5(R)-5-ethyl-5-methyl-4-(4-(methylsulfonyl)phenyl)-3-(2-propoxy)-5H-furan-2-one;

5,5-dimethyl-3-(2,2-dimethylpropyloxy)4-(4-(methylsulfonyl)phenyl)-5H-furan-2-one;

5(R)-3-(1-cyclopropyl-ethoxy)-5-ethyl-5-methyl(4-(methyl sulfonyl)phenyl-5H-furan-2-one;

5(S)-5-ethyl-5-methyl-4-(4-(methylsulfonyl)phenyl-3-(2-propoxy)-5H-furan-2-one;

3-(1-cyclopropylethoxy)-5,5-dimethyl-4-(4-(methylsulfonyl)phenyl)-5H-furan-2-one;

3-(1-cyclopropylethoxy)-5,5-dimethyl-4-(4-(methylsulfonyl)phenyl)-5H-furan-2-one;

5,5-dimethyl-3-(isobutoxy)-4-(4-(methylsulfonyl)phenyl)-5H-furan-2-one;

3-(4-bromophenoxy)-5,5-dimethyl-4-(4-(methylsulfonyl)phenyl)-5H-furan-2-one;

3-(2-quinolinoxy)-5,5-dimethyl-4-(4-(methylsulfonyl)phenyl)-5H-furan-2-one;

3-(2-chloro-5-pyridyloxy)-5,5-dimethyl-4-(4-(methylsulfonyl)phenyl)-5H-furan-2-one;

3-(6-benzothiazolyloxy)-5,5-dimethyl-4-(4-(methylsulfonyl)phenyl)-5H-furan-2-one;

3-(6-chloro-2-pyridyloxy)-5,5-dimethyl-4-(4-(methylsulfonyl)phenyl)-5H-furan-2-one;

3-(4-quinazolyloxy)-5,5-dimethyl-4-(4-(methylsulfonyl)phenyl)-5H-furan-2-one;

(5R)-3-(5-fluoro-2-pyridyloxy)-5-ethyl-5-methyl-4-(4-methylsulfonyl)phenyl-5H-furan-2-one;

(5R)-3-(4-fluorophenoxy)-5-ethyl-5-methyl-4-(4-methylsulfonyl)phenyl-5H-furan-2-one;

(5R)-3-(5-fluoro-2-pyridyloxy)-5-methyl-4-(4-methylsulfonyl)phenyl-5-(2,2,2-trifluoroethyl)-5H-furan-2-one;

3-(1-isoquinolinyloxy)-5,5-dimethyl-4-(methylsulfonyl)phenyl-5H-furan-2-one;

(5R)-3-(4-fluorophenoxy)-5-methyl-4-(4-methylsulfonyl)phenyl-5-(2,2,2-trifluoroethyl)-5H-furan-2-one;

3-(3-fluoro-2-pyridyloxy)-5,5-dimethyl-4-(methylsulfonyl)phenyl-5H-furan-2-one;

(5R)-3-(3,4-difluorophenoxy)-5-methyl-4-(4-methylsulfonyl)phenyl-5-(2,2,2-trifluoroethyl)-5H-furan-2-one;

(5R)-3-(5-chloro-2-pyridyloxy)-5-ethyl-5-methyl-4-(methylsulfonyl)phenyl-5H-furan-2-one;

3-(3,4-difluorophenoxy)-5-methyl-5-trifluoromethyl-4-(4-methylsulfonyl)phenyl-5H-furan-2-one;

3-(3,4-difluorophenoxy)-5-methyl-4-(4-(methylsulfonyl)phenyl)-5-propyl-5H-furan-2-one;

3-cyclobutyloxy-5,5-dimethyl-4-(4-methylsulfonylphenyl-5H-furan-2-one;

3-(1-indanyloxy)-5,5-dimethyl-4-(4-(methylsulfonyl)phenyl)-5H-furan-2-one;

3-(2-indanyloxy)-5,5-dimethyl-4-(4-methylsulfonyl)phenyl)-5H-furan-2-one;

3-cyclopentyloxy-5,5-dimethyl-4-(4-methylsulfonylphenyl)5H-furan-2-one;

3-(3,3-dimethylcyclopentyloxy)-5,5-dimethyl-4-(4-methylsulfonyl-phenyl)-5H-furan-2-one;

3-isopropoxy-5-methyl-4-(4-methylsulfonylphenyl)-5-propyl-5H-furan-2-one;

3-(2-methoxy-5-pyridyloxy)-5,5-dimethyl-4-(4-methylsulfonyl)phenyl-5H-furan-2-one;

3-(5-methyl-2-pyridyloxy)-5,5-dimethyl-4-(4-methylsulfonyl)phenyl-5H-furan-2-one;

(5RS)-3-(3,4-difluorophenoxy)-5-methyl-4-(4-methylsulfonyl)phenyl-5-(2,2,2-trifluoroethyl)-5H-furan-2-one;

3-(3-chloro-4-methoxyphenoxy)-5,5-dimethyl-4-(4-methylsulfonyl)phenyl-5H-furan-2-one;

(5R)-3-(3-chloro-4-methoxyphenoxy)-5-ethyl-5-methyl-4-(4-methylsulfonyl)phenyl-5H-furan-2-one;

(5R)-3-(4-chlorophenoxy)-5-trifluoroethyl-5-methyl-4-(4-methylsulfonyl)phenyl-5H-furan-2-one;

(5R)-3-(4-bromophenoxy)-5-trifluoroethyl-5-methyl-4-(4-methylsulfonyl)phenyl-5H-furan-2-one;

5-cyclopropylmethyl-3-(3,4-difluorophenoxy)-5-methyl-(4-methylsulfonyl)phenyl-5H-furan-2-one;

(5R)-3-(3-fluorophenoxy)-5-ethyl-5-methyl-4-(4-methylsulfonyl)phenyl-5H-furan-2-one;

(5R)-3-(4-chloro-3-fluorophenoxy)-5-ethyl-5-methyl-4-(4-methylsulfonyl)phenyl-5H-furan-2-one;

(5R)-3-phenoxy-5-ethyl-5-methyl-4-(4-methylsulfonyl)phenyl-5H-furan-2-one;

(5R)-3-(4-chloro-3-methylphenoxy)-5-ethyl-5-methyl-4-(4-methylsulfonyl)phenyl-5H-furan-2-one;

3-(4-chloro-3-methylphenoxy)-5-5-dimethyl-4-(4-methylsulfonyl)phenyl-5H-furan-2-one;

(5R)-3-(5-bromo-2-pyridyloxy)-4-(4-methylsulfonylphenyl)-5-methyl-5-(2,2,2-trifluoroethyl)-5H-furan-2-one;

(5R)-3-(5-bromo-2-pyridyloxy)-4-(4-methylsulfonylphenyl)-5-ethyl-5-methyl-5H-furan-2-one;

3-(5-chloro-6-methyl-2-pyridyloxy)-5,5-dimethyl-4-(4-methylsulfonyl)phenyl-5H-furan-2-one;

3-(5-cyclopropyl-2-pyridyloxy)-5,5-dimethyl-4-(4-methylsulfonyl)phenyl-5H-furan-2-one;

3-(1-cyclopropylethoxy)-4-(4-methylsulfonyl)phenyl-5H-furan-2-one; and

3-(cyclopropylmethoxy)4-(4-methylsulfonyl)phenyl-5H-furan-2-one;

or a pharmaceutically acceptable salt or stereoisomer thereof.

Inhibitors of COX-2 that are particularly useful in the instant method of treatment are:

3-phenyl-4-(4-(methylsulfonyl)phenyl)-2-(5H)-furanone; and

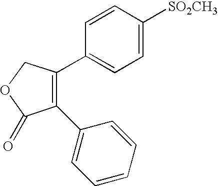

5-chloro-3-(4-methylsulfonyl)phenyl-2-(2-methyl-5-pyridinyl)pyridine;

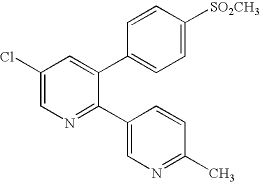

or a pharmaceutically acceptable salt thereof.

General and specific synthetic procedures for the preparation of the COX-2 inhibitor compounds described above are found in U.S. Pat. No. 5,474,995, issued Dec. 12, 1995, U.S. Pat. No. 5,861,419, issued Jan. 19, 1999, and U.S. Pat. No. 6,001,843, issued Dec. 14, 1999, all of which are herein incorporated by reference.

Compounds that have been described as specific inhibitors of COX-2 and are therefore useful in the present invention include, but are not limited to, the following:

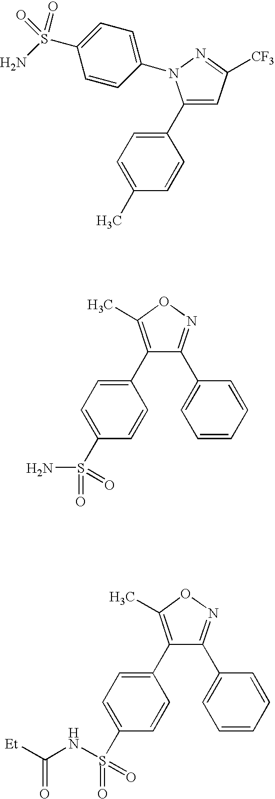

or a pharmaceutically acceptable salt thereof.

Compounds which are described as specific inhibitors of COX-2 and are therefore useful in the present invention, and methods of synthesis thereof, can be found in the following patents, pending applications and publications, which are herein incorporated by reference: WO 94/15932, published Jul. 21, 1994, U.S. Pat. No. 5,344,991, issued Jun. 6, 1994, U.S. Pat. No. 5,134,142, issued Jul. 28, 1992, U.S. Pat. No. 5,380,738, issued Jan. 10, 1995, U.S. Pat. No. 5,393,790, issued Feb. 20, 1995, U.S. Pat. No. 5,466,823, issued Nov. 14, 1995, U.S. Pat. No. 5,633,272, issued May 27, 1997, and U.S. Pat. No. 5,932,598, issued Aug. 3, 1999.

Compounds which are specific inhibitors of COX-2 and are therefore useful in the present invention, and methods of synthesis thereof, can be found in the following patents, pending applications and publications, which are herein incorporated by reference: U.S. Pat. No. 5,474,995, issued Dec. 12, 1995, U.S. Pat. No. 5,861,419, issued Jan. 19, 1999, U.S. Pat. No. 6,001,843, issued Dec. 14, 1999, U.S. Pat. No. 6,020,343, issued Feb. 1, 2000, U.S. Pat. No. 5,409,944, issued Apr. 25, 1995, U.S. Pat. No. 5,436,265, issued Jul. 25, 1995, U.S. Pat. No. 5,536,752, issued Jul. 16, 1996, U.S. Pat. No. 5,550,142, issued Aug. 27, 1996, U.S. Pat. No. 5,604,260, issued Feb. 18, 1997, U.S. Pat. No. 5,698,584, issued Dec. 16, 1997, and U.S. Pat. No. 5,710,140, issued Jan. 20, 1998.

Other examples of angiogenesis inhibitors include, but are not limited to, endostation, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]methyl]-1H-1,2,3-triazole-4-carboxamide, CM101, squalamine, combretastatin, RPI4610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the αᵥβ₃ integrin, to compounds which selectively antagonize, inhibit or counter-act binding of a physiological ligand to the αvβ5 integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁ and α₆β₄ integrins.

Some specific examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BEBX1382, 2,3,9,10,11,12-hexahydro-10-(hydroxymethyl)-10-hydroxy-9-methyl-9,12-epoxy-1H-diindolo[1,2,3-fg:3′,2′,1′-kl]pyrrolo[3,4-i][1,6]benzodiazocin-1-one, SH268, genistein, STI571, CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, ST1571A, N-4-chlorophenyl-4-(4-pyridylmethyl)-1-phthalazinamine, and EMD121974.

The instant compounds are also useful, alone or in combination with platelet fibrinogen receptor (GP IIb/IIIa) antagonists, such as tirofiban, to inhibit metastasis of cancerous cells. Tumor cells can activate platelets largely via thrombin generation. This activation is associated with the release of VEGF. The release of VEGF enhances metastasis by increasing extravasation at points of adhesion to vascular endothelium. Amirkhosravi, Platelets 10:285-292 (1999). Therefore, the present compounds can serve to inhibit metastasis, alone or in combination with GP IIb/IIIa antagonists. Examples of other fibrinogen receptor antagonists include abciximab, eptifibatide, sibrafiban, lamifiban, lotrafiban, cromofiban, and CT50352.

If formulated as a fixed dose, such combination products employ the compounds of this invention within the dosage range described below and the other pharmaceutically active agent(s) within its approved dosage range. Compounds of the instant invention may alternatively be used sequentially with known pharmaceutically acceptable agent(s) when a combination formulation is inappropriate.

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

The present invention also encompasses a pharmaceutical composition useful in the treatment of cancer, comprising the administration of a therapeutically effective amount of the compounds of this invention, with or without pharmaceutically acceptable carriers or diluents. Suitable compositions of this invention include aqueous solutions comprising compounds of this invention and pharmacologically acceptable carriers, e.g., saline, at a pH level, e.g., 7.4. The solutions may be introduced into a patient's bloodstream by local bolus injection.

When a compound according to this invention is administered into a human subject, the daily dosage will normally be determined by the prescribing physician with the dosage generally varying according to the age, weight, and response of the individual patient, as well as the severity of the patient's symptoms.

In one exemplary application, a suitable amount of compound is administered to a mammal undergoing treatment for cancer. Administration occurs in an amount between about 0.1 mg/kg of body weight to about 60 mg/kg of body weight per day, preferably of between 0.5 mg/kg of body weight to about 40 mg/kg of body weight per day.

Definitions

The compounds of the present invention may have asymmetric centers, chiral axes, and chiral planes (as described in: E. L. Eliel and S. H. Wilen, Stereochemistry of Carbon Compounds, John Wiley & Sons, New York, 1994, pages 1119-1190), and occur as racemates, racemic mixtures, and as individual diastereomers, with all possible isomers and mixtures thereof, including optical isomers, being included in the present invention. In addition, the compounds disclosed herein may exist as tautomers and both tautomeric forms are intended to be encompassed by the scope of the invention, even though only one tautomeric structure is depicted. For example, any claim to compound A below is understood to include tautomeric structure B, and vice versa, as well as mixtures thereof.

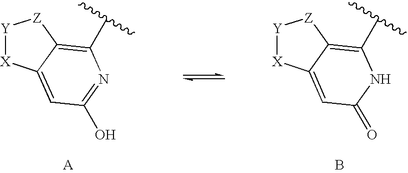

When any variable occurs more than one time in any constituent, its definition on each occurrence is independent at every other occurrence. Also, combinations of substituents and variables are permissible only if such combinations result in stable compounds. Lines drawn into the ring systems from substituents mean that the indicated bond may be attached to any of the substitutable ring atoms. If the ring system is polycyclic, it is intended that the bond be attached to any of the suitable atoms on the proximal ring only.

In the structure

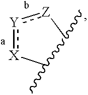

it is understood that a and b can be a single bond or a double bond provided that a and b are not a double bond at the same time. Therefore, the following structures are encompassed by the instant invention:

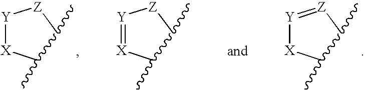

As defined in the claims X, Y, and Z are C, S, N or O provided that at least one of X, Y and Z is C.

Therefore,

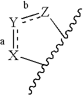

includes, but is not limited to the following:

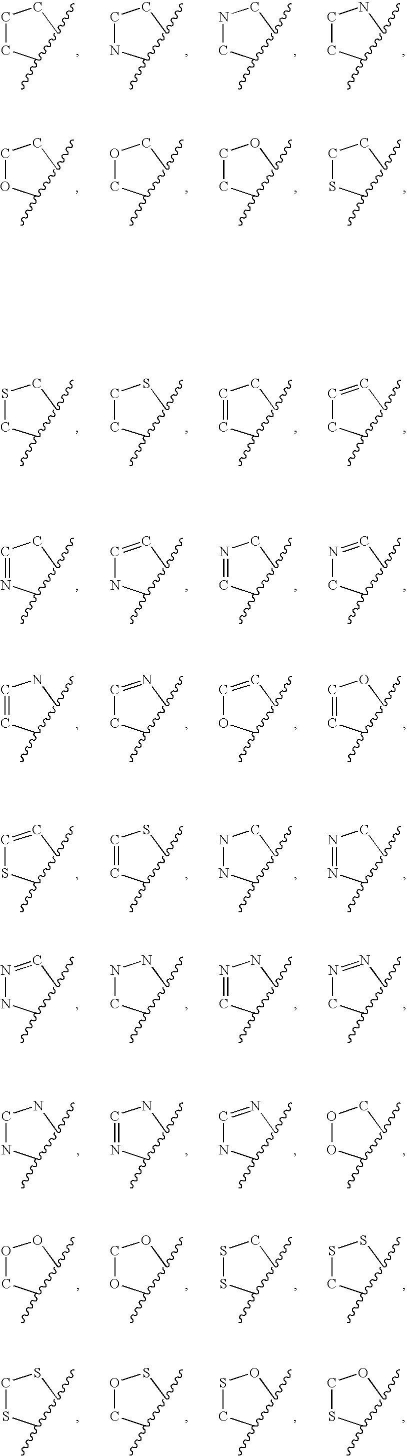

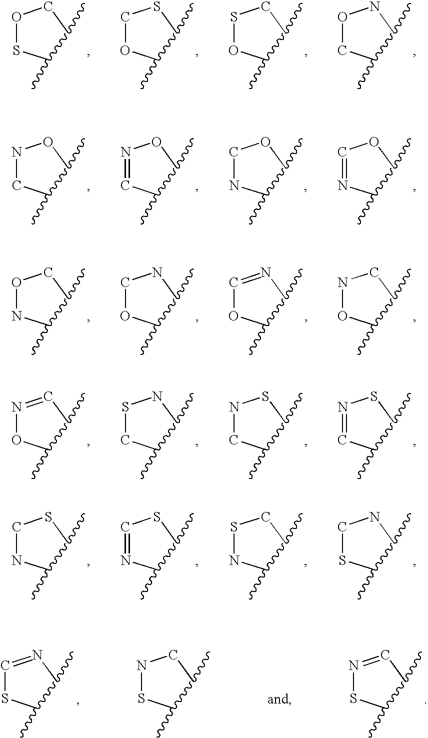

Attachment of the R⁵ substituent to the above described structures can occur via a carbon atom or via any heteroatom. Moreover, attachment of more than one R⁵ substituent may occur at any carbon atom or any heteroatom. Therefore the following are possible substitution patterns:

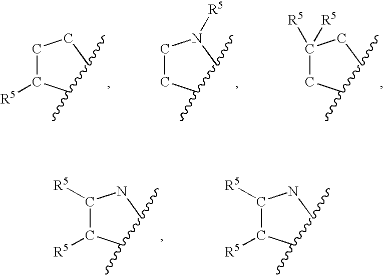

and so on.

Also, where R⁵ is oxo it is understood that R⁵ is:

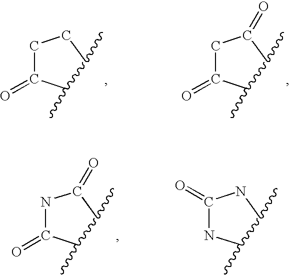

and so on. It is understood that in the case of multiple R⁵'s, each R⁵ can vary independently. Thus, if n=2 and one R⁵ is SO₂Me and one R⁵ is Br, the following are possible structures:

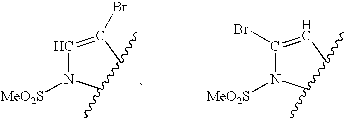

and so on.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same atom or on different atoms, so long as a stable structure results. The phrase “optionally substituted with one or more substituents” should be taken to be equivalent to the phrase “optionally substituted with at least one substituent” and in such cases the preferred embodiment will have from zero to three substituents.

As used herein, “alkyl” is intended to include both branched, straight-chain, and cyclic saturated aliphatic hydrocarbon groups having the specified number of carbon atoms. For example, C₁-C₁₀, as in “C₁-C₁₀ alkyl” is defined to include groups having 1, 2, 3, 4, 5, 6, 7, 8, 9 or 10 carbons in a linear or branched, arrangement and may be cyclic or acyclic. For example, “C₁-C₁₀ alkyl” specifically includes methyl, ethyl, propyl, butyl, pentyl, hexyl, heptyl, octyl, nonyl, decyl, and so on, as well as cyclo-alkyls such as cyclopropyl, cyclobutyl, cyclopentyl, cyclohexyl, tetrahydronaphthalene, methylenecylohexyl, and so on. In some instances, definitions may appear for the same variable reciting both alkyl and cycloalkyl when a different number of carbons is intended for the respective substituents. The use of both terms in one definition should not be interpreted to mean in another definition that “alkyl” does not encompass “cycloalkyl” when only “alkyl” is used.

“Alkoxy” represents an alkyl group of indicated number of carbon atoms as defined above attached through an oxygen bridge.

If no number of carbon atoms is specified, the term “alkenyl” refers to a non-aromatic hydrocarbon radical, which may be branched or unbranched and cyclic or acyclic, containing from 2 to 10 carbon atoms and at least one carbon to carbon double bond. Preferably one carbon to carbon double bond is present, and up to four non-aromatic carbon-carbon double bonds may be present. Thus, “C₂-C₆ alkenyl” means an alkenyl radical having from 2 to 6 carbon atoms. Alkenyl groups include ethenyl, propenyl, butenyl, 2-methylbutenyl, cyclohexenyl, methylenylcyclohexenyl, and so on.

The term “alkynyl” refers to a hydrocarbon radical, which may be branched or unbranched and cyclic or acyclic, containing from 2 to 10 carbon atoms and at least one carbon to carbon triple bond. Up to three carbon-carbon triple bonds may be present. Thus, “C₂-C₆ alkynyl” means an alkynyl radical having from 2 to 6 carbon atoms. Alknyl groups include ethynyl, propynyl, butynyl, 3-methylbutynyl and so on.

In certain instances, substituents may be defined with a range of carbons that includes zero, such as (C₀-C₆)alkylene-aryl. If aryl is taken to be phenyl, this definition would include phenyl itself as well as —CH₂Ph, —CH₂CH₂Ph, CH(CH₃)CH₂CH(CH₃)Ph, and so on.

As used herein, “aryl” is intended to mean phenyl and substituted phenyl, including moieties with a fused benzo group. Examples of such aryl elements include phenyl, naphthyl, tetrahydro-naphthyl, indanyl, biphenyl, phenanthryl, anthryl or acenaphthyl. In cases where the aryl substituent is bicyclic, it is understood that attachment is via the phenyl ring. Unless otherwise indicated, “aryl” includes phenyls substituted with one or more substituents.

The term heteroaryl, as used herein, represents a stable monocyclic or bicyclic ring of up to 7 atoms in each ring, wherein at least one ring is aromatic and contains from 1 to 4 heteroatoms selected from the group consisting of O, N and S. Heteroaryl groups within the scope of this definition include but are not limited to: acridinyl, carbazolyl, cinnolinyl, quinoxalinyl, pyrrazolyl, indolyl, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. As with the definition of heterocycle below, “heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring, respectively.

As appreciated by those of skill in the art, “halo” or “halogen” as used herein is intended to include chloro, fluoro, bromo and iodo.

The term “heterocycle” or “heterocyclyl” as used herein is intended to mean a 5- to 10-membered aromatic or nonaromatic heterocycle containing from 1 to 4 heteroatoms selected from the group consisting of O, N and S, and includes bicyclic groups. “Heterocyclyl” therefore includes the above mentioned heteroaryls, as well as dihydro and tetrathydro analogs thereof. Further examples of “heterocyclyl” include, but are not limited to the following: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, aziridynyl, 1,4-dioxanyl, hexahydroazepinyl, piperazinyl, piperidinyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof. Attachment of a heterocyclyl substituent can occur via a carbon atom or via a heteroatom.

The alkyl, alkenyl, alkynyl, cycloalkyl, aryl, heteroaryl and heterocyclyl substituents may be substituted or unsubstituted, unless specifically defined otherwise. For example, a (C₁-C₆)alkyl may be substituted with one or more substituents selected from OH, oxo, halogen, alkoxy, dialkylamino, or heterocyclyl, such as morpholinyl, piperidinyl, and so on. In this case, if one substituent is oxo and the other is OH, the following are included in the definition: (C═O)CH₂CH(OH)CH₃, —(C═O)OH, —CH₂(OH)CH₂CH(O), and so on.

The pharmaceutically acceptable salts of the compounds of this invention can be synthesized from the compounds of this invention which contain a basic or acidic moiety by conventional chemical methods. Generally, the salts of the basic compounds are prepared either by ion exchange chromatography or by reacting the free base with stoichiometric amounts or with an excess of the desired salt-forming inorganic or organic acid in a suitable solvent or various combinations of solvents. For example, such conventional non-toxic salts include those derived from inorganic acids such as hydrochloric, hydrobromic, sulfuric, sulfamic, phosphoric, nitric and the like: and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like. Similarly, the salts of the acidic compounds are formed by reactions with the appropriate inorganic or organic base.

Preferably W is C or N. More preferably W is C.

Preferably R¹ is CN or phenyl. More preferably R¹ is CN.

Preferably R², R³ and R⁴ are H.

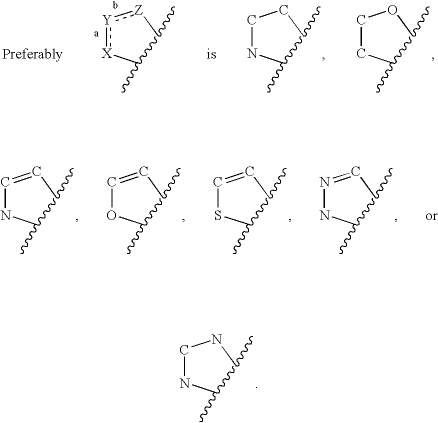

### Schemes

The compounds of the instant invention may be prepared from the general reaction schemes for the preparation of heterocyclic analogs as shown in Schemes A-D.

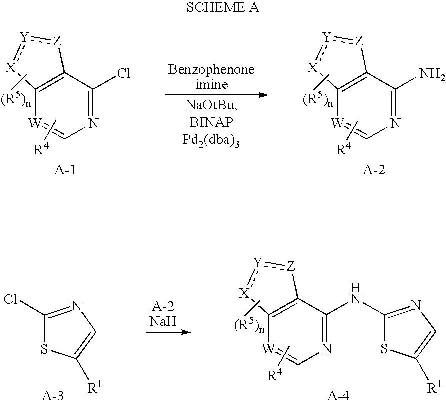

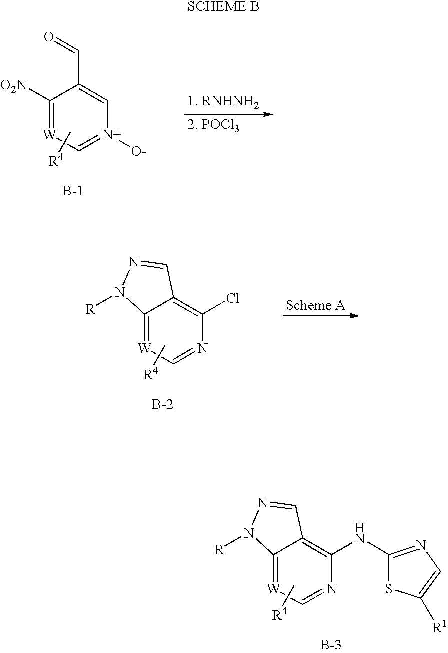

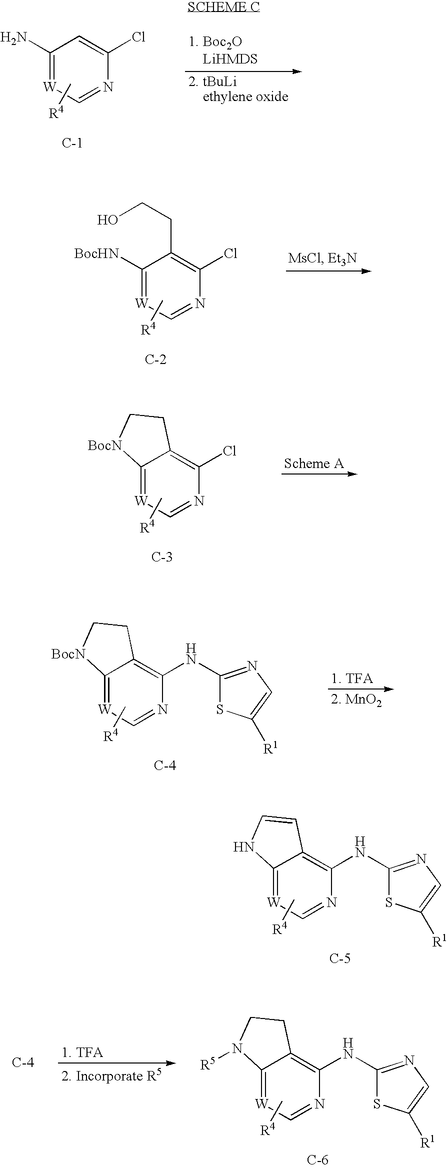

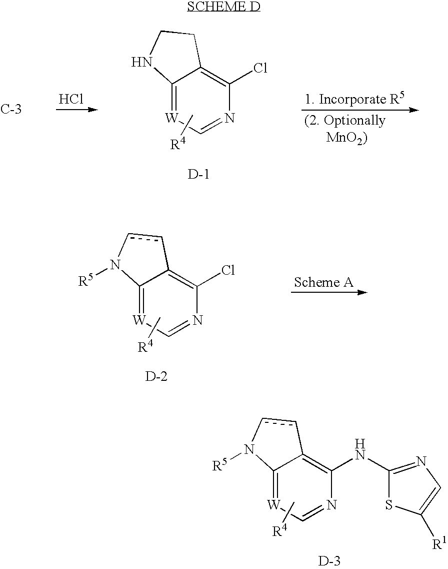

### Assays

The compounds of the instant invention described in the Examples were tested by the assays described below and were found to have kinase inhibitory activity. Other assays are known in the literature and could be readily performed by those of skill in the art. (see, for example, Dhanabal et al., Cancer Res. 59:189-197; Xin et al., J. Biol. Chem. 274:9116-9121; Sheu et al., Anticancer Res. 18:44354441; Ausprunk et al., Dev. Biol. 38:237-248; Gimbrone et al., J. Natl. Cancer Inst. 52:413-427; Nicosia et al., In Vitro 18:538-549).

### I. VEGF Receptor Kinase Assay

VEGF receptor kinase activity is measured by incorporation of radio-labeled phosphate into polyglutamic acid, tyrosine, 4:1 (pEY) substrate. The phosphorylated pEY product is trapped onto a filter membrane and the incorporation of radio-labeled phosphate quantified by scintillation counting.

### Materials

VEGF Receptor Kinase

The intracellular tyrosine kinase domains of human KDR (Terman, B. I. et al., Oncogene 6:1677-1683 (1991)) and Flt-1 (Shibuya, M. et al., Oncogene 5:519-524 (1990)) were cloned as glutathione S-transferase (GST) gene fusion proteins. This was accomplished by cloning the cytoplasmic domain of the KDR kinase and the Flt-1 kinase domain as an in frame fusion at the carboxy terminus of the GST gene. Soluble recombinant GST-kinase domain fusion protein and recombinant GST-Flt-1 kinase domain fusion protein were expressed in Spodoptera frugiperda (Sf21) insect cells (Invitrogen) using a baculovirus expression vector (pAcG2T, Pharmingen).

The other materials used and there compositions were as follows:

Lysis buffer: 50 mM Tris pH 7.4, 0.5 M NaCl, 5 mM DTT, 1 mM EDTA, 0.5% triton X-100, 10% glycerol, 10 μg/mL of each leupeptin, pepstatin and aprotinin and 1 mM phenylmethylsulfonylfluoride (PMSF) (all Sigma).

Wash buffer: 50 mM Tris pH 7.4, 0.5 M NaCl, 5 mM DTT, 1 mM EDTA, 0.05% triton X-100, 10% glycerol, 10 μg/mL of each leupeptin, pepstatin and aprotinin and 1 mM PMSF.

Dialysis buffer: 50 mM Tris pH 7.4, 0.5 M NaCl, 5 mM DIT, 1 mM EDTA, 0.05% triton X-100, 5% glycerol, 10 μg/mL of each leupeptin, pepstatin and aprotinin and 1 nM PMSF.

10× reaction buffer: 200 mM Tris, pH 7.4, 1.0 M NaCl, 50 mM MnCl₂, 10 mM DTT and 5 mg/mL bovine serum albumin (Sigma).

Enzyme dilution buffer: 50 mM Tris, pH 7.4, 0.1 M NaCl, 1 mM DTT, 10% glycerol, 100 mg/mL BSA.

10× Substrate: 750 μg/mL poly (glutamic acid, tyrosine; 4:1) (Sigma).

Stop solution: 30% trichloroacetic acid, 0.2 M sodium pyrophosphate (both Fisher).

Wash solution: 15% trichloroacetic acid, 0.2 M sodium pyrophosphate.

Filter plates: Millipore #MAFC NOB, GF/C glass fiber 96 well plate.

### Method

A. Protein Purification

1. Sf21 cells were infected with recombinant virus at a multiplicity of infection of 5 virus particles/cell and grown at 27° C. for 48 hours.

2. All steps were performed at 4° C. Infected cells were harvested by centrifugation at 1000×g and lysed at 4° C. for 30 minutes with 1/10 volume of lysis buffer followed by centrifugation at 100,000×g for 1 hour. The supernatant was then passed over a glutathione Sepharose column (Pharmacia) equilibrated in lysis buffer and washed with 5 volumes of the same buffer followed by 5 volumes of wash buffer. Recombinant GST-KDR and GST-Flt-1 kinase domain proteins were eluted with wash buffer/10 mM reduced glutathione (Sigma) and dialyzed against dialysis buffer.

B. VEGF Receptor Kinase Assay

1. Add 5 μl of inhibitor or control to the assay in 50% DMSO.

2. Add 35 μl of reaction mix containing 5 μl of 10× reaction buffer, 5 μl 25 mM ATP/10 μCi [³³P]ATP (Amersham), and 5 μl 10× substrate.

3. Start the reaction by the addition of 10 μl of KDR (25 nM) in enzyme dilution buffer.

4. Mix and incubate at room temperature for 15 minutes.

5. Stop by the addition of 50 μl stop solution.

6. Incubate for 15 minutes at 4° C.

7. Transfer a 90 μl aliquot to filter plate.

8. Aspirate and wash 3 times with wash solution.

Add 30 μl of scintillation cocktail, seal plate and count in a Wallac Microbeta scintillation counter.

C. FLT-1 Kinase Assay

Flt-1 was expressed as a GST fusion to the Flt-1 kinase domain and was expressed in baculovirus/insect cells. The following protocol was employed to assay compounds for Flt-1 kinase inhibitory activity:

1. Inhibitors were diluted to account for the final dilution in the assay, 1:20.

2. The appropriate amount of reaction mix was prepared at room temperature:

10× Buffer (20 mM Tris pH 7.4/0.1 M NaCl/1 mM DTT final)

0.1M MnCl₂ (5 mM final)

pEY substrate (75 μg/mL)

ATP/[³³P]ATP (2.5 i/1 μCi final)

BSA (500 μg/mL final).

3. 5 μL of the diluted inhibitor was added to the reaction mix. (Final volume of 5 μL in 50% DMSO). To the positive control wells, blank DMSO (50%) was added.

4. 35 μL of the reaction mix was added to each well of a 96 well plate.

5. Enzyme was diluted into enzyme dilution buffer (kept at 4° C.). 10 μL, of the diluted enzyme was added to each well and mix (5 nM final).

6. To the negative control wells, 10 μL 0.5 M EDTA was added per well instead (final 100 mM).

7. Incubation was then carried out at room temperature for 30 minutes.

8. Stopped by the addition of an equal volume (50 μL) of 30% TCA/0.1M Na pyrophosphate.

9. Incubation was then carried out for 15 minutes to allow precipitation.

10. Transfered to Millipore filter plate.

11. Washed 3× with 15% TCA/0.1M Na pyrophosphate (125 μL per wash).

12. Allowed to dry under vacuum for 2-3 minutes.

13. Dryed in hood for ˜20 minutes.

14. Assembled Wallac Millipore adapter and added 50 μL of scintiliant to each well and counted.

II. Human Umbilical Vein Endothelial Cell Mitogenesis Assay

Human umbilical vein endothelial cells (HUVECs) in culture proliferate in response to VEGF treatment and can be used as an assay system to quantify the effects of KIDR kinase inhibitors on VEGF stimulation. In the assay described, quiescent HUVEC monolayers are treated with vehicle or test compound 2 hours prior to addition of VEGF or basic fibroblast growth factor (bFGF). The mitogenic response to VEGF or bFGF is determined by measuring the incorporation of [³H]thymidine into cellular DNA.

### Materials

HUVECs: HUVECs frozen as primary culture isolates are obtained from Clonetics Corp. Cells are maintained in Endothelial Growth Medium (EGM; Clonetics) and are used for mitogenic assays described in passages 3-7 below.

Culture Plates: NUNCLON 96-well polystyrene tissue culture plates (NUNC #167008).

Assay Medium: Dulbecco's modification of Eagle's medium containing 1 g/mL glucose (low-glucose DMEM; Mediatech) plus 10% (v/v) fetal bovine serum (Clonetics).

Test Compounds: Working stocks of test compounds are diluted serially in 100% dimethylsulfoxide (DMSO) to 400-fold greater than their desired final concentrations. Final dilutions to 1× concentration are made directly into Assay Medium immediately prior to addition to cells.

10× Growth Factors: Solutions of human VEGF165 (500 ng/mL; R&D Systems) and bFGF (10 ng/mL; R&D Systems) are prepared in Assay Medium.

10× [³H]Thymidine: [Methyl-³H]thymidine (20 Ci/mmol; Dupont-NEN) is diluted to 80 μCi/mL in low-glucose DMEM.

Cell Wash Medium: Hank's balanced salt solution (Mediatech) containing 1 mg/mL bovine serum albumin (Boehringer-Mannheim).

Cell Lysis Solution: 1 N NaOH, 2% (w/v) Na₂CO₃.

### Method

1. HUVEC monolayers maintained in EGM are harvested by trypsinization and plated at a density of 4000 cells per 100 μL Assay Medium per well in 96-well plates. Cells are growth-arrested for 24 hours at 37° C. in a humidified atmosphere containing 5% CO₂.

2. Growth-arrest medium is replaced by 100 μL Assay Medium containing either vehicle (0.25% [v/v] DMSO) or the desired final concentration of test compound. All determinations are performed in triplicate. Cells are then incubated at 37° C. with 5% CO₂ for 2 hours to allow test compounds to enter cells.

3. After the 2-hour pretreatment period, cells are stimulated by addition of 10 μL/well of either Assay Medium, 10×VEGF solution or 10×bFGF solution. Cells are then incubated at 37° C. and 5% CO₂.

4. After 24 hours in the presence of growth factors, 10× [³H]thymidine (10 μL/well) is added.

5. Three days after addition of [³H]thymidine, medium is removed by aspiration, and cells are washed twice with Cell Wash Medium (400 μL/well followed by 200 μL/well). The washed, adherent cells are then solubilized by addition of Cell Lysis Solution (100 μL/well) and warming to 37° C. for 30 minutes. Cell lysates are transferred to 7-mL glass scintillation vials containing 150 μL of water. Scintillation cocktail (5 ml/vial) is added, and cell-associated radioactivity is determined by liquid scintillation spectroscopy.

Based upon the foregoing assays the compounds of Formula I are inhibitors of VEGF and thus are useful for the inhibition of angiogenesis, such as in the treatment of ocular disease, e.g., diabetic retinopathy and in the treatment of cancers, e.g., solid tumors. The instant compounds inhibit VEGF-stimulated mitogenesis of human vascular endothelial cells in culture with IC₅₀ values between 0.001-5.0 μM. These compounds may also show selectivity over related tyrosine kinases (e.g., FGFR1 and the Src family; for relationship between Src kinases and VEGFR kinases, see Eliceiri et al., Molecular Cell 4:915-924 (1999)).

### EXAMPLES

Examples provided are intended to assist in a further understanding of the invention. Particular materials employed, species and conditions are intended to be further illustrative of the invention and not limiting of the reasonable scope thereof. The compounds of this invention may be prepared by employing reactions as shown in the following schemes, in addition to other standard manipulations that are known in the literature.

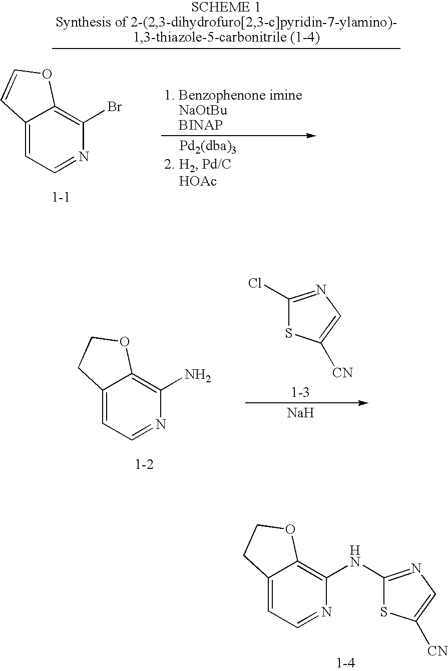

2,3-dihydrofuro[2,3-c]pyridin-7-amine (1-2)

To a solution of 7-bromofuro[2,3-c]pyridine (88 mg, 0.44 mmole), benzophenone imine (0.09 mL, 0.53 mmole), NaOtBu (60 mg, 0.62 mmole), and racemic BINAP (16 mg, 0.03 mmole) in dry toluene (1 mL) was added Pd₂(dba)₃ (8 mg, 0.01 mmole) and the mixture heated to 80° C. After 18 hours, the mixture was cooled to room temperature, diluted with Et₂O, filtered through a pad of Celite®, and concentrated. The residue was taken up in MeOH (2 mL) and hydroxylamine (0.1 mL of a 50% solution in H₂O) was added at room temperature. After 2 hours, the mixture was concentrated. Flash column chromatography (100% EtOAc) gave a pale yellow solid which was sufficiently pure for use in the next step. A solution of this solid (57 mg, 0.425 mmole) in HOAc (3 mL) was hydrogenated under balloon pressure H₂ with 10% Pd/C (50 mg). After 72 hours, the mixture was filtered through a pad of Celite® and concentrated. The residue was taken up in saturated NaHCO₃ and extracted with CH₂Cl₂ (3×). The combined organic layers were dried (MgSO₄), filtered, and concentrated to a pale yellow solid: ¹H-NMR (300 MHz, CDCl₃) δ 7.59 (d, J=5.2 Hz, 1H), 6.62 (d, J=5.2 Hz, 1H), 4.60 (t, J=8.9 Hz, 2H), 3.20 (t, J=8.9 Hz, 2H), 2.54 (bs, 2H).

2-(2,3-dihydrofuro[2,3-c]pyridin-7-ylamino)-1,3-thiazole-5-carbonitrile (1-4)

To a solution of 2,3-dihydrofuro[2,3-c]pyridin-7-amine (54 mg, 0.4 mmole) in dry THF (3 mL) was added NaH (50 mg, 60% suspension in mineral oil, 1.19 mmole) and 2-chloro-5-cyanothiazole (1-3, 86 mg, 0.6 mmole). The mixture was heated to reflux. After 2 hours, the mixture was cooled to room temperature, quenched with H₂O, and extracted with CH₂Cl₂ (4×). The combined organic layers were dried (MgSO₄), filtered, and concentrated. Trituration with Et₂O gave the title compound as a pale yellow solid: ¹H-NMR (300 MHz, d₆-DMSO) δ 11.96 (bs, 1H), 8.24 (s, 1H), 7.92 (d, J=4.9 Hz, 1H), 7.09 (d, J=4.9 Hz, 1H), 4.69 (t, J=9.2 Hz, 2H), 3.30 (t, J=9.2 Hz, 2H); MS (ES) (M+H)⁺ 245.

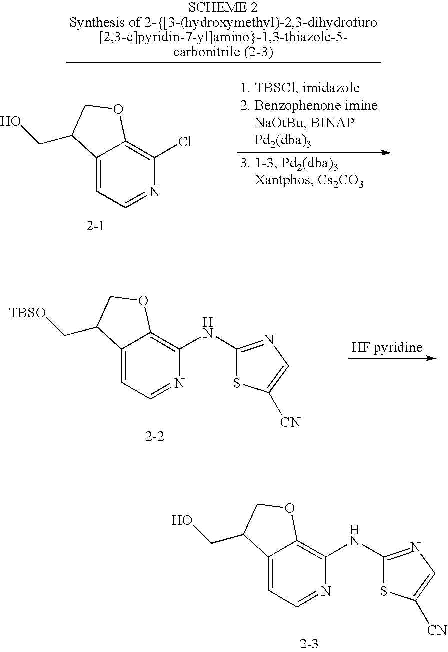

2-{[3-({[tert-butyl(dimethyl)silyl]oxy}methyl)-2,3-dihydrofuro[2,3-c]pyridin-7-yl]amino}-1,3-thiazole-5-carbonitrile (2-2)

To a solution of (7-chloro-2,3-dihydrofuro[2,3-c]pyridin-3-yl)methanol (B. Joseph; A. Benarab; G. Guillaumet Heterocycles 1995, 41, 2769-2776, 300 mg, 1.62 mmole) in dry DMF (5 mL) was added imidazole (132 mg, 1.94 mmole) and TBDMSCl (292 mg, 1.94 mmole) at room temperature. After 18 hours, the mixture was concentrated. The residue was taken up in CH₂Cl₂, filtered, and concentrated. Flash column chromatography (10% EtOAc/hexanes) gave a clear oil which solidified under vacuum. A solution of this oil (465 mg, 1.55 mmole), benzophenone imine (0.31 mL, 1.86 mmole), NaOtBu (209 mg, 2.17 mmole), racemic BINAP (58 mg), and Pd₂(dba)₃ (28 mg) in dry toluene (8 mL) was heated to 80° C. After 18 hours the mixture was cooled to room temperature, diluted with Et₂O, filtered though a pad of Celite®, and concentrated. The residue was taken up in MeOH (8 mL) and hydroxylamine (0.32 mL of a 50% solution in H₂O) was added at room temperature.

After 5 hours, the mixture was concentrated. Flash column chromatography (gradient, 60-100% EtOAc/hexanes) gave a pale yellow solid. A solution of this solid (122 mg, 0.44 mmole), 2-chloro-5-cyanothiazole (1-3,76 mg, 0.52 mmole), Cs₂CO₃ (198 mg, 0.61 mmole), Pd₂(dba)₃ (8 mg), and Xantphos (9,9-dimethyl-4,5-bis (diphenylphosphino)xanthene, 15 mg) in dry dioxane (3 mL) was heated to reflux. After 18 hours, the mixture was cooled to room temperature, diluted with H₂O, and extracted with CH₂Cl₂ (3×). The combined organic layers were dried (MgSO₄), filtered, and concentrated. Trituration with Et₂O gave tan solid: ¹H-NMR (300 MHz, CDCl₃) δ 9.41 (bs, 1H), 7.98 (s, 1H), 7.96 (d, J=4.9 Hz, 11), 6.97 (d, J=4.9 Hz, 1H), 4.77 (m, 1H), 4.60 (m, 1H), 3.77 (m, 3H), 0.88 (s, 9H), 0.01 (s, 6H).

2-{[3-(hydroxymethyl)-2,3-dihydrofuro[2,3-c]pyridin-7-yl]amino}-1,3-thiazole-5-carbonitrile (2-3)

To a suspension of 2-{[3-({[tert-butyl(dimethyl)silyl]oxy}methyl)-2,3-dihydrofuro[2,3-c]pyridin-7-yl]amino}-1,3-thiazole-5-carbonitrile (2-2, 146 mg, 0.38 mmole) in dry TBF (3 mL) was added 0.3 mL HF-pyridine at room temperature. After 2 hours, the mixture was diluted with H₂O and neutralized with solid K₂CO₃. The phases were separated and the aqueous extracted with CH₂Cl₂ (3×). The combined organic layers were dried (MgSO₄), filtered, and concentrated. Flash column chromatography (gradient, 2-5% MeOH/CHCl₃) gave the title compound as a pale yellow solid: ¹H-NMR (300 MHz, d₆-DMSO) δ 11.96 (bs, 1H), 8.24 (s, 1H), 7.93 (d, J=4.9 Hz, 1H), 7.12 (d, J=4.9 Hz, 1H), 5.03 (t, J=4.9 Hz, 1H), 4.75 (t, J=9.2 Hz, 1H) 4.54 (m, 1H), 3.65 (m, 2 I); MS (ES) (M+H)⁺ 275.

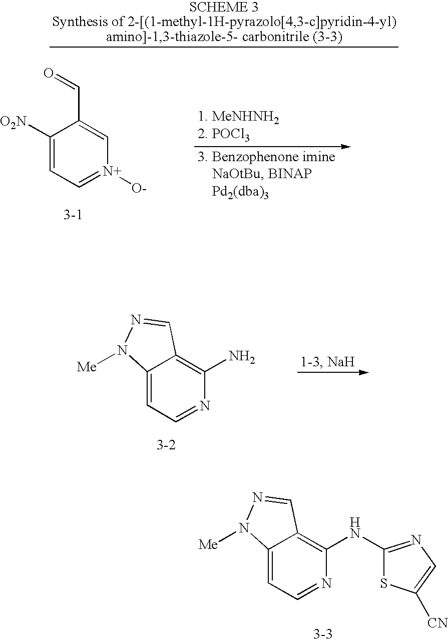

1-methyl-1H-pyrrolo[3,2-c]pyridin-4-amine (3-2)

To a suspension of 4-nitronicotinaldehyde 1-oxide (S. F. Wnuk; E. Lewandowska; C. A. Valdez; S. Kinastowski Tetrahedron 2000, 56; 7667-7672, 3-1, 268 mg, 1.59 mmole) in EtOH (8 mL) was added N-methylhydrazine (0.25 mL, 4.78 mmole) and the mixture was heated to reflux. After 3 hours, the mixture was cooled to room temperature and concentrated. Flash column chromatography (10% MeOH/CH₂Cl₂) gave an orange solid: ¹H-NMR (300 MHz, d₆-DMSO) δ 8.78 (s, 1H), 8.10 (s, 21H), 7.80 (d, J=6.4 Hz, 1H), 4.06 (s, 3H).

This product (110 mg, 0.74 mmole) was taken up in POCl₃ (3 mL) and heated to reflux. After 2 hours, the mixture was cooled to room temperature and concentrated to dryness. The residue was taken up in saturated NaHCO₃ and extracted with CH₂Cl₂ (3×). The combined organic layers were dried (MgSO₄), filtered, and concentrated to a tan solid. Examination of the crude product by ¹H-NMR indicates a 6:1 mixture of the 4- and 6-chloro isomers which were carried on to the next step without purification. A solution of these chloride isomers (105 mg, 0.63 mmole), benzophenone imine (0.13 mL, 0.75 mmole), NaOtBu (84 mg, 0.88 mmole), Pd₂(dba)₃ (12 mg) and racemic BINAP (23 mg) in dry toluene (3 mL) was deoxygenated (3× pump/N2) then heated to 80° C. After 2 hours, additional benzophenone imine (0.13 mL, 0.75 mmole), NaOtBu (84 mg, 0.88 mmole), Pd₂(dba)₃ (12 mg) and racemic BINAP (23 mg) were added and heating continued. After 18 hours, the mixture was cooled to room temperature, diluted with Et₂O, filtered through a pad of Celite®, and concentrated. The residue was taken up in MeOH (3 mL) and hydroxylamine (0.2 mL of a 50% solution in H₂O) was added. After 4 hours, the mixture was concentrated. Flash column chromatography (10% MeOH/CH₂Cl₂) gave a pale yellow solid: ¹H-NMR (300 MHz, CDCl₃) δ 7.91 (s, 1H), 7.86 (d, J=6.4 Hz, 1H), 6.71 (d, J=6.1 Hz, 1H), 4.99 (bs, 2H), 4.00 (s, 3H).

2-[(1-methyl-1H-pyrazolo[4,3-c]pyridin-4-yl)amino]-1,3-thiazole-5-carbonitrile (3-3)

To a solution of 1-methyl-1H-pyrrolo[3,2-c]pyridin-4-amine (3-2, 27 mg, 0.18 mmole) in dry THF (2 mL) was added NaH (20 mg, 60% dispersion in mineral oil, 0.45 mmole) at room temperature. After gas evolution had ceased 2-chloro-5-cyanothiazole (40 mg, 0.27 mmole) was added and the mixture heated to reflux. After 3 hours, the mixture was cooled to room temperature and diluted with H₂O. The resulting solid was collected by filtration, washed with H₂O, and dried in vacuo. Trituration with Et₂O/hexanes gave the title compound: ¹H-NMR (300 MHz, d₆-DMSO) δ 12.89 (s, 1H), 8.59 (s, 1H), 8.36 (s, 1H), 8.14 (d, J=6.1 Hz, 1H), 7.37 (d, J=6.1 Hz, 1H), 4.06 (s, 3H); MS (ES) (M+H)⁺ 256.

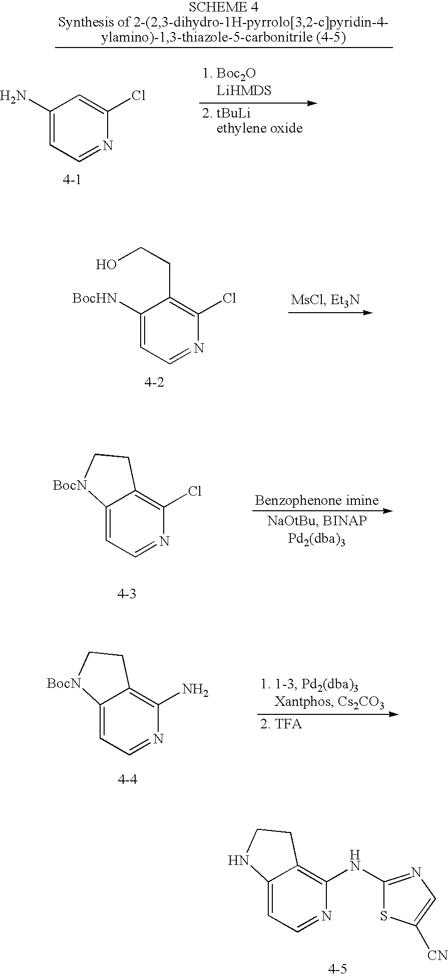

tert-butyl-2-chloro-3-(2-hydroxyethyl)pyridin-4-ylcarbamate (4-2)

To a solution of 4-amino-2-chloropyridine (4-1, 1.0 g, 7.78 mmole) and di-tert-butyl-di-carbonate (1.7 g, 7.78 mmole) in dry THF (20 mL) was added LHMDS (15.6 mL, 1M in THF, 15.6 mmole) slowly at 0° C. After 30 minutes, the mixture was warmed to room temperature, quenched with saturated NH₄Cl, and extracted with EtOAc (3×). The combined organic layers were dried (MgSO₄), filtered, and concentrated. Flash column chromatography (20% EtOAc/hexanes) gave tert-butyl 2-chloropyridin-4-ylcarbamate as a white solid: ¹H-NMR (300 MHz, CDCl₃) δ 8.20 (d, J=6.0 Hz, 1H), 7.50 (s, 1H), 7.15 (dd, J=2.0 Hz and J=4.0 Hz, 1H), 6.75 (bs, 1H), 1.50 (s, 9H).

To a solution of tert-butyl-2-chloropyridin-4-ylcarbamate (1.44 g, 6.3 mmole) in dry THF (20 mL) was added tBuLi (9.3 mL, 1.7M in pentane, 15.74 mmole) dropwise at −78° C. After 1 hour, ethylene oxide (approx 0.75 mL) was condensed in cold, dry THF (2 mL) and transfered to the lithiated chloropyridine. After 15 minutes the mixture was warmed to 40° C. After 30 minutes, the bath was removed and the mixture allowed to warm to room temperature. After 1 hour, the mixture was quenched with saturated NH₄Cl and extracted with EtOAc (3×). The combined organic layers were dried (MgSO₄), filtered, and concentrated. Flash column chromatography (20% EtOAc/hexanes) gave the title compound as a white solid:

¹H-NMR (300 MHz, CDCl₃) δ 8.40 (bs, 1H), 8.15 (d, J=5.5 Hz, 1H), 7.90 (d, J=5.5 Hz, 1H), 4.05 (m, 2H), 3.05 (t, J=8.6 Hz, 2H), 1.50 (s, 9H).

tert-butyl-4-amino-2,3-dihydro-1H-pyrrolo[3,2-c]pyridine-1-carboxylate (4-3)

To a solution of tert-butyl-2-chloro-3-(2-hydroxyethyl)pyridin-4-ylcarbamate (4-2,586 mg, 2.15 mmole) and Et₃N (0.66 mL, 4.73 mmole) in CH₂Cl₂ (10 mL) was added MsCl (0.18 mL, 2.36 mmole) dropwise at −5° C. The mixture was allowed to warm as the bath warmed to room temperature. After 18 hours, additional Et₃N (0.66 mL) was added and the mixture heated to reflux. After 1 hour, the mixture was cooled to room temperature and diluted with H₂O. The layers were separated and the aqueous extracted with CH₂Cl₂ (2×). The combined organic layers were dried (MgSO₄), filtered, and concentrated. Flash column chromatography (10% EtOAc/hexanes) gave the title compound as a white solid: ¹H-NMR (300 MHz, CDCl₃) δ 8.15 (d, J=5.5 Hz, 1H), 7.50 (bs, 1H), 4.05 (t, J=8.6 Hz, 2H), 3.10 (t, J=8.6 Hz, 2H), 1.50 (s, 9H).

tert-butyl-4-amino-2,3-dihydro-1H-pyrrolo[3,2-c]pyridine-1-carboxylate (4-4)

To a solution of tert-butyl-4-amino-2,3-dihydro-1H-pyrrolo[3,2-c]pyridine-1-carboxylate (4-3, 200 mg, 0.79 mmole), benzophenone imine (0.16 mL, 0.94 mmole), NaOtBu (106 mg, 1.1 mmole), and racemic BINAP (29 mg) in dry toluene (3 mL) was added Pd₂(dba)₃ (14 mg) and the mixture heated to 80° C. After 18 hours, additional racemic BINAP (29 mg) and Pd₂(dba)₃ (14 mg) was added. After 2 hours, the mixture was cooled to room temperature, diluted with Et₂O, filtered through a pad of Celite®, and concentrated. The residue was taken up in MeOH (3 mL) and hydroxylamine (1.6 mL of a 50% solution in H₂O) was added at room temperature. After 18 hours, the mixture was concentrated. Flash column chromatography (gradient, 0-10% MeOH(CH₂Cl₂) gave the title compound as a white solid: ¹H-NMR (300 MHz, CDCl₃) δ 7.90 (d, J=5.5 Hz, 1H), 7.40 (bs, 1H), 4.20 (bs, 2H), 4.05 (t, J=8.6 Hz, 2H), 2.90 (t, J=8.6 Hz, 2H), 1.50 (s, 9H).

2-(2,3-dihydro-1H-pyrrolo[3,2-c]pyridin-4-ylamino)-1,3-thiazole-5-carbonitrile (4-5)

A solution of tert-butylamino-2,3-dihydro-1H-pyrrolo[3,2-c]pyridine-1-carboxylate (4-4,76 mg, 0.32 mmole), 2-chloro-5-cyanothiazole (56 mg, 0.39 mmole), Cs₂CO₃ (147 mg, 0.45 mmole), Pd₂(dba)₃ (6 mg), and Xantphos (11 mg) in dry dioxane (3 mL) was heated to reflux. After 18 hours, the mixture was cooled to room temperature, diluted with H₂O, and extracted with EtOAc (3×). The combined organic layers were dried (MgSO₄), filtered, and concentrated. Flash column chromatography (gradient, 35-50% EtOAc/hexanes) gave a light tan solid: ¹H-NMR (300 MHz, CDCl₃) δ 8.20 (d, J=5.5 Hz, 1H), 8.10 (bs, 1H), 7.90 (s, 1H), 7.40 (bs, 1H), 4.15 (t, J=8.6 Hz, 2H), 3.05 (t, J=8.6 Hz, 2H), 1.6 (s, 9H); MS (ES) (M+H)⁺ 344.

To a suspension of this solid (65 mg, 0.19 mmole) in 25% DMS/CH₂Cl₂ (2 mL) was added 2 mL TFA at room temperature. After 1 hours, the mixture was concentrated. The residue was taken up in saturated NaHCO₃ and extracted with EtOAc (3×). The combined organic layers were dried (MgSO₄), filtered, and concentrated. Flash column chromatography (60% EtOAc/hexanes) gave the title compound as a pale yellow solid: ¹H-NMR (300 MHz, d₆-DMSO) δ 11.50 (s, 1H), 8.20 (s, 1H), 7.82 (d, J=5.5 Hz, 1H), 6.54 (s, 1H), 6.24 (d, J=5.5 Hz, 1H), 3.56 (t, J=8.6 Hz, 2H), 3.01 (t, J=8.6 Hz, 2H); MS (ES) (M+H)+244.

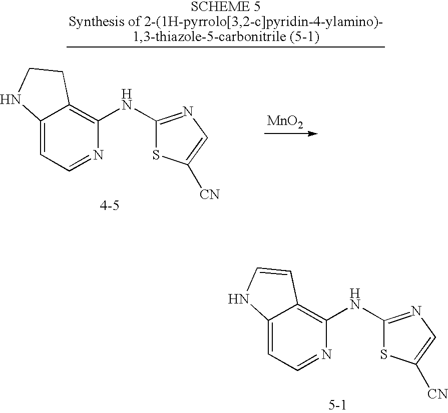

2-(1H-pyrrolo[3,2-c]pyridin-4-ylamino)-1,3-thiazole-5-carbonitrile (5-1)

To a solution of 2-(2,3-dihydro-1H-pyrrolo[3,2-c]pyridin-4-ylamino)-1,3-thiazole-5-carbonitrile (4-5, 50 mg, 0.21 mmole) in dry THF (1.5 mL) was added MnO₂ (179 mg, 2.1 mmole) and the mixture heated to reflux. After 3 hours, the mixture was cooled to room temperature, filtered through a pad of Celite®, and concentrated. Flash column chromatography (40% EtOAc/hexanes) gave the title compound as an off-white solid: ¹H-NMR (300 MHz, d₆-DMSO) δ 12.37 (s, 1H), 11.68 (s, 1H), 8.30 (s, 11]), 7.96 (d, J=5.8 z, 1H), 7.40 (m, 1H), 7.14 (m, 2H); MS (ES) (M+H)⁺ 242.

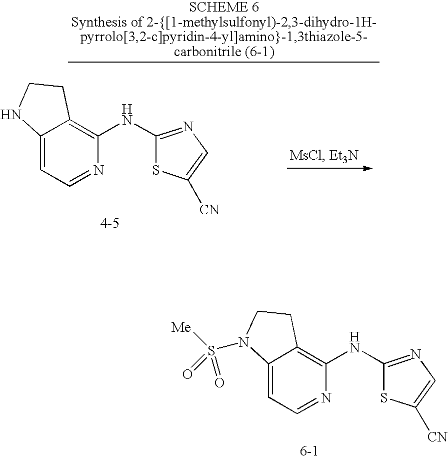

2-{[1-(methylsulfonyl)-2,3-dihydro-1H-pyrrolo[3,2-c]pyridin-4-yl]amino}-1,3-thiazole-5-carbonitrile (6-1)

To a solution of 2-(2,3-dihydro-1H-pyrrolo[3,2-c]pyridin-4-amino)-1,3-thiazole-5-carbonitrile (4-5, 50 mg, 0.21 mmole) in dry THF (1.5 mL) was added pyridine (0.02 mL, 0.23 mmole) then MsCl (0.02 mL, 0.23 mmole) at 0° C. After 30 minutes the mixture was warmed to room temperature. After 18 hours, Et₃N (0.04 mL, 0.27 mmole) was added and stirring continued. After 3 hours, the mixture was diluted with H₂O. The resulting solid was collected by filtration and air dried. Flash column chromatography (gradient, 50-100% EtOAc/hexanes) gave a yellow solid. Purification by reverse phase HPLC (5-100% CH₃CN/H₂O+0.1% TFA) gave the TFA salt of title compound as an off-white solid: ¹H-NMR (300 MHz, d₆-DMSO) δ 11.96 (bs, 1H) 8.29 (bs, 1H), 8.21 (d, J=5.2 Hz, 1H), 7.00 (d, J=5.2 Hz, 1H), 4.04 (m, 2H), 3.20 (m, 2H) 3.13 (s, 3H); MS (ES) (M+H)⁺ 322.

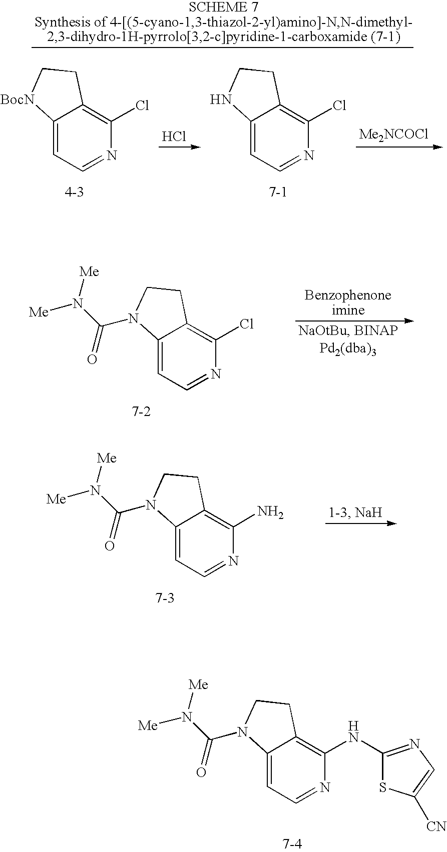

4-chloro-2,3-dihydro-1H-pyrrolo[3,2-c]pyridine (7-1)

To a solution of tert-butyl-4-chloro-2,3-dihydro-1H-pyrrolo[3,2-c]pyridine-1-carboxylate (4-3,686 mg, 2.69 mmole) in CH₂Cl₂ (5 mL) was added 4N HCl in dioxane (15 mL) at room temperature. After 4 hours TFA (10 mL) was added. After 18 hours, the mixture was concentrated. The resulting oil was taken up in saturated NaHCO₃. The resulting white solid was collected by filtration, washed with H₂O, and dried in vacuo. ¹H-NMR (300 MHz, CDCl₃) δ 7.90 (d, J=5.4 Hz, 1 H), 6.38 (d, J=5.4 Hz, 1H), 4.30 (bs, 1H), 3.70 (t, J=8.6 Hz, 2H), 3.10 (t, J=8.6 Hz, 2H).

4-chloro-N,N-dimethyl-2,3-dihydro-1H-pyrrolo[3,2-c]pyridine-1-carboxamide (5-2)

To a suspension of 4-chloro-2,3-dihydro-1H-pyrrolo[3,2-c]pyridine (7-1, 100 mg, 0.65 mmole) in CH₂Cl₂ (3 mL) was added Et₃N (0.14 mL, 0.97 mmole) then Me₂NCOCl (0.08 mL, 0.84 mmole) at room temperature. After 18 hours reaction the mixture was heated to reflux. After 18 hours, the mixture was diluted with H₂O and extracted with CH₂Cl₂ (4×). The combined organic layers were dried (MgSO₄), filtered, and concentrated. Flash column chromatography (40% EtOAc/hexanes) gave the title compound as a pale yellow solid: ¹H-NMR (500 MHz, CDCl₃) δ 8.10 (d, J=5.4 Hz, 1H), 6.77 (d, J=5.4 Hz, 1H), 4.00 (t, J=8.6 Hz, 2H), 3.11 (t, J=8.6 Hz, 2H), 2.96 (s, 6H).

4-amino-N,N-dimethyl-2,3-dihydro-1H-pyrrolo[3,2-c]pyridine-1-carboxamide (7-3)

A solution of 4-chloro-N,N-dimethyl-2,3-dihydro-1H-pyrrolo[3,2-c]pyridine-1-carboxamide (7-2, 102 mg, 0.45 mmole), benzophenone imine (0.09 mL, 0.54 mmole), NaOtBu (61 mg, 0.63 mmole), Pd₂(dba)₃ (8 mg) and racemic BINAP (17 mg) in dry toluene (3 mL) was degassed (3× pump/N2) then heated to 80° C. After 5 hours, the mixture was cooled to room temperature and concentrated. The residue was taken up in MeOH (3 mL) and hydroxylamine (0.1 mL of a 50% solution in H₂O) was added. After 18 hours, the mixture was concentrated. Flash column chromatography (gradient, 5-10% MeOH/CH₂Cl₂) gave an orange foam: ¹H-NMR (500 MHz, CDCl₃) δ 7.79 (d, J=5.9 Hz, 1H), 6.39 (d, J=5.9 Hz, 1H), 4.86 (bs, 2H), 4.01 (t, J=8.6 Hz, 2H), 2.95 (s, 6H), 2.88 (t, J=8.6 Hz, 2H).

4-[(5-cyano-1,3-thiazol-2-yl)amino]-N,N-dimethyl-2,3-dihydro-1H-pyrrolo[3,2-c]pyridine-1-carboxamide (7-4)

To a solution of 4-amino-N,N-dimethyl-2,3-dihydro-1H-pyrrolo[3,2-c]pyridine-1-carboxamide (7-3,75 mg, 0.36 mmole) in dry THF (3 mL) was added NaH (40 mg, 60% dispersion in mineral oil, 0.91 mmole). After gas evolution had ceased 2-chloro-5-cyanothiazole (79 mg, 0.55 mmole) was added and the mixture heated to reflux. After 3 hours, the mixture was cooled to room temperature, diluted with H₂O, and extracted with CH₂Cl₂ (4×). The combined organic layers were dried (MgSO₄), filtered, and concentrated. Trituration with Et₂O gave the title compound as a yellow solid: ¹H-NMR (500 MHz, d₆-DMSO) δ 11.80 (bs, 1H) 8.25 (s, 1H), 8.09 (d, J=5.6 Hz, 1H), 6.69 (d, J=5.6 Hz, 1H), 3.92 (t, J=8.3 Hz, 2H), 3.11 (t, J=8.3 Hz, 2H), 2.88 (s, 6H), MS (ES) (M+H)⁺ 315.

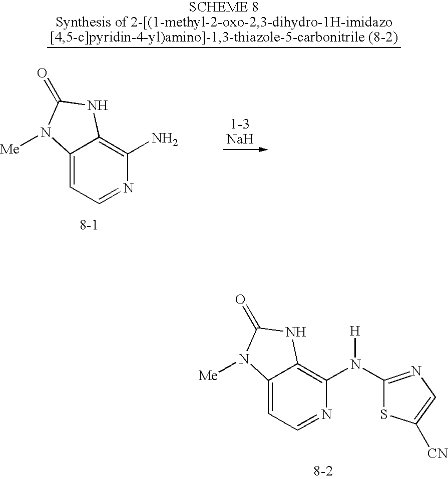

2-[(1-methyl-2-oxo-2,3-dihydro-1H-imidazo[4,5-c]pyridin-4-yl)amino]-1,3-thiazole-5-carbonitrile (8-2)

4-Amino-1-methyl-1,3-dihydro-2H-imidazo[4,5-c]pyridin-2-one (0.053 g, 0.32 mmol) was dissolved in 2 mL THF. 2-Chloro-1,3-thiazole-5-carbonitrile (0.056 g, 0.39 mmol) and sodium hydride (60% dispersion in mineral oil) (0.039 g, 1.61 mmol) were added and the solution was heated to 75° C. After 7 hours, more sodium hydride (0.039 g, 1.61 mmol) was added. After 24 hours, the solution was allowed to cool to room temperature. H₂O was added and the reaction was concentrated in vacuo (to remove THF). 1N HCl was added to adjust to neutral pH. The resulting precipitate was filtered and washed with water to afford a dark brown solid. The solid was purified by reverse phase chromatography (gradient, 5-100% CH₃CN/H₂O+0.1% TFA). The fractions containing the desired compound were concentrated to dryness to afford the free base. Free base: ¹H NMR ([MSO-d₆) δ 11.58 (bs, 1H), 10.24 (bs, 1H), 8.29 (s, 1H), 8.08 (d, 1H, J=5.37 Hz), 7.06 (d, 1H, J=5.37 Hz), 2.54 (s, 1H).

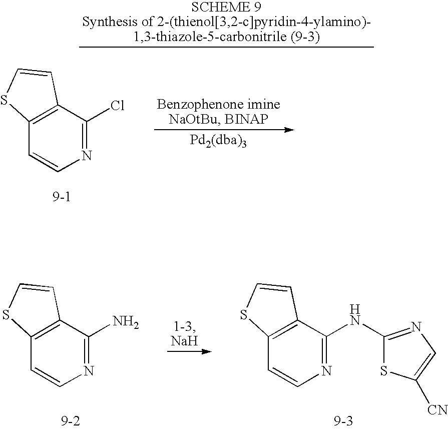

thieno[3,2-c]pyridin-4-amine (9-2)

To a solution of 4-chlorothieno[3,2-c]pyridine (9-1, J. New, W. Christopher, et al J. Med. Chem. 32 1989 1147-1156, 0.110 g, 0.65 mmol) in anhydrous toluene (2 mL) under N2 was added racemic BINAP (0.024 g, 0.04 mmol), Pd₂(dba)₃ (0.012 g, 0.01 mmol), and sodium tert-butoxide (0.087 g, 0.90 mmol). Benzophenone imine (0.130 mL, 0.77 mmol) was added and the reaction was heated to 80° C. After 1.5 hours, the reaction mixture was cooled to room temperature, diluted with ether, filtered through celite, and washed with ether. The filtrate was concentrated to afford an orange oil. The oil was dissolved in MeOH (5 mL) and treated with hydroxylamine (50% aq) (0.059 mL, 1.94 mmol). The solution was stirred at room temperature. After 19 hours, more hydroxylamine (50% aq) (0.059 mL, 1.94 mmol) was added. After 21 hours, the reaction was concentrated to afford an orange oil. The oil was purified by flash column chromatography (100% EtOAc). The fractions containing the desired compound were concentrated to dryness to afford thieno[3,2-c]pyridin-4-amine as a light yellow solid. Free base: ¹H NMR (DMSO-d₆) δ 7.75 (d, 1H, J=5.80 Hz), 7.64 (d, 1H, J=5.49 Hz), 7.56 (d, 1H, J=5.49 Hz), 7.12 (d, 1H, J=5.80 Hz), 6.55 (bs, 2H).

2-(thieno[3,2-c]pyridin-4-ylamino)-1,3-thiazole-5-carbonitrile (9-3)

Thieno[3,2-c]pyridin-4-amine (7-2, 0.055 g, 0.36 mmol) was dissolved in 2 mL THF. 2-Chloro-1,3-thiazole-5-carbonitrile (0.063 g, 0.44 mmol) and sodium hydride (60% dispersion in mineral oil) (0.035 g, 1.45 mmol) were added and the solution was heated to 75° C. After 4.5 hours, the solution was allowed to cool to room temperature. H₂O was added and the reaction was concentrated in vacuo (to remove THF). 1N HCl was added to adjust to neutral pH. The resulting precipitate was filtered and washed with water to afford a yellow solid. The solid was purified by flash column chromatography (40:60 EtOAc/hexanes). The fractions containing the desired compound were concentrated to dryness to afford the free base as a light yellow solid. Free base: ¹H NMR (DMSO-d₆) δ 12.65 (bs, 1H), 8.36 (s, 1H), 8.25-8.18 (m, 2H), 7.91 (d, 1H, J=5.80 Hz), 7.78 (d, 1H, J=5.19 Hz). [M+H]+259.0109.

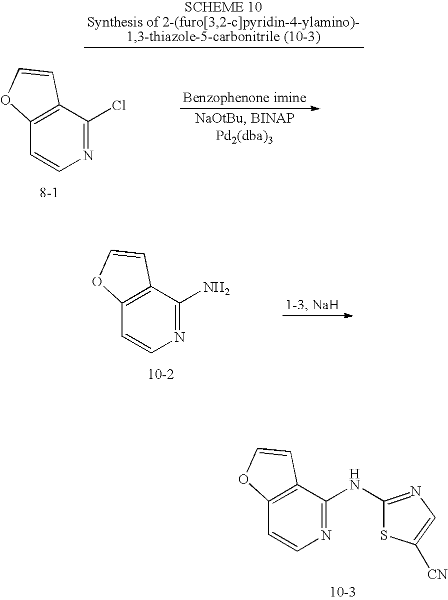

furo[3,2-c]pyridin-4-amine (10-2)

To a solution of 4-chlorofuro[3,2-c]pyridine (10-1, J. New, W. Christopher, et al, J. Med. Chem. 32 1989 1147-1156., 0.079 g, 0.51 mmol) in anhydrous toluene (2 mL) under N2 was added racemic BINAP (0.019 g, 0.03 mmol), Pd₂(dba)₃ (0.009 g, 0.01 mmol), and sodium tert-butoxide (0.069 g, 0.72 mmol). Benzophenone imine (0.103 mL, 0.61 mmol) was added and the reaction was heated to 80° C. After 3 hours, the reaction mixture was cooled to room temperature, diluted with ether, filtered through celite, and washed with ether. The filtrate was concentrated to afford an orange oil. The oil was dissolved in MeOH (7 mL) and treated with hydroxylamine (50% aq) (0.047 mL, 1.54 mmol). The solution was stirred at room temperature for 20 hours and the reaction was concentrated to afford an orange oil. The oil was purified by flash column chromatography (100% EtOAc). The fractions containing the desired compound were concentrated to dryness to furo[3,2-c]pyridin-4-amine as a light yellow solid. Free base: ¹H NMR (DMSO-d₆) δ 7.80 (d, 1H, J=2.14 Hz), 7.75 (d, 1H, J=5.80 Hz), 7.03 (d, 1H, J=1.22 Hz), 6.79 (d, 1H, J=5.80 Hz), 6.45 (bs, 2H).

2-(furo[3,2-c]pyridin-4-amino)-1,3-thiazole-5-carbonitrile (10-3)

Furo[3,2-c]pyridin-4-amine (10-2, 0.043 g, 0.32 mmol) was dissolved in 2 mL THF. 2-Chloro-1,3-thiazole-5-carbonitrile (0.056 g, 0.39 mmol) and sodium hydride (60% dispersion in mineral oil) (0.031 g, 1.29 mmol) were added and the solution was heated to 75° C. After 3.5 hours, the solution was allowed to cool to room temperature. H₂O was added and the reaction was concentrated in vacuo (to remove THF). 1N HCl was added to adjust to neutral pH. The resulting precipitate was filtered and washed with water to afford an orange solid. The solid was purified by flash column chromatography (20:80 EtOAc/hexanes). The fractions containing the desired compound were concentrated to dryness to afford the free base as a light yellow solid. Free base: ¹H NMR (DMSO-d₆) δ 12.69 (bs, 1H), 8.34 (s, 1H), 8.27 (d, 1H, J=6.10 Hz), 8.11 (d, 1H, J=2.14 Hz), 7.52 (bs, 1H), 7.43 (d, 1H, J=5.80 Hz). [M+H]+=243.0334.

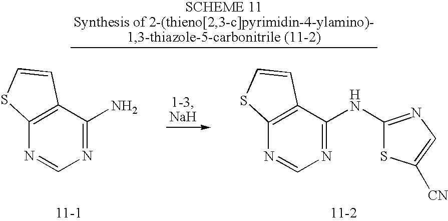

2-(thieno[2,3-d]pyrimidin-4-4]amino)-1,3-thiazole-5-carbonitrile (11-2)

Thieno[2,3-d]pyrimidin-4-amine (11-1, 0.032 g, 0.21 mmol) was dissolved in 2 mL THF. 2-Chloro-1,3-thiazole-5-carbonitrile (0.037 g, 0.25 mmol) and sodium hydride (60% dispersion in mineral oil) (0.020 g, 0.85 mmol) were added and the solution was heated to 75° C. After 3.5 hours, the solution was allowed to cool to room temperature. H₂O was added and the reaction was concentrated in vacuo (to remove THF). 1N HCl was added to adjust to neutral pH. The resulting precipitate was filtered and washed with water to afford a yellow solid. The solid was purified by flash column chromatography (gradient, 20-40% EtOAc/hexanes). The fractions containing the desired compound were concentrated to dryness to afford the free base as a light yellow solid which was then purified by reverse phase chromatography (gradient, 5-100% CH₃CN/H₂O+0.1% TFA). The fraction containing the desired compound was concentrated to dryness to afford the free base as a white solid. Free base: ¹H NMR (DMSO-d₆) δ 13.15 (bs, 1H), 8.87 (s, 1H), 8.45 (s, 1H), 8.10 (d, 1H, J=5.37 Hz), 7.92 (d, 1H, J=6.10 Hz). [M+H]+=260.0.

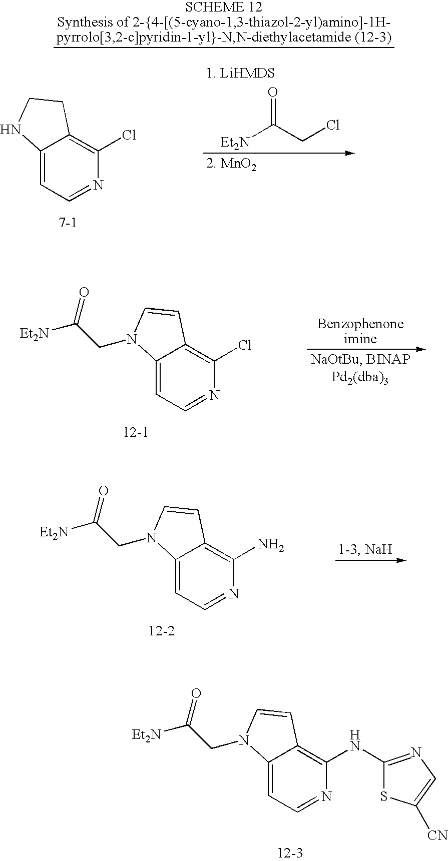

2-(4-chloro-1H-pyrrolo[3,2-c]pyridin-1-yl]-N,N-diethylacetamide (12-1)

To a solution of 4-chloro-2,3-dihydro-1H-pyrrolo[3,2-c]pyridine (7-1, 140 mg, 0.91 mmole) in dry THF (5 mL) was added LHMDS (1.1 mL, 1M in THF, 1.09 mmole) slowly at 0° C. After 20 minutes, N,N-diethyl-2-chloroacetamide (0.17 mL, 1.27 mmole) was added. The mixture was allowed to warm to room temperature as the bath warmed. After 18 hours, the mixture was diluted with H₂O and extracted with EtOAc (3×). The combined organic layers were dried (MgSO₄), filtered, and concentrated. Flash column chromatography (70% EtOAc/hexanes) gave 2-(4-chloro-2,3-dihydro-1H-pyrrolo[3,2-c]pyridin-1-yl)-N,N-diethylacetamide as an off-white solid: ¹H-NMR (500 MHz, CDCl₃) δ 7.90 (d, J=5.37 Hz, 1H), 6.11 (d, J=5.37 Hz, 1H), 3.97 (s, 2H), 3.71 (t, J=8.79 Hz, 2H), 3.39 (q, J=7.08 Hz, 2H), 3.31 (q, J=7.08 Hz, 2H), 3.11 (t, J=8.79 Hz, 2H), 1.24 (t, J=7.08 Hz, 31H), 1.14 (t, J=7.32 Hz, 3H).

To a solution of 2-(4-chloro-2,3-dihydro-1H-pyrrolo[3,2-c]pyridin-1-yl)-N,N-diethylacetamide (184 mg, 0.69 mmole) in dry THF (5 mL) was added MnO₂ (300 mg, 3.43 mmole) and the mixture was heated to reflux. After 2 hours, additional MnO₂ (300 mg) was added and heating continued. After 18 hours, additional MnO₂ (300 mg) was added and heating continued. After 8 hours, additional MnO₂ (300 mg) was added and heating continued. After 18 hours, the mixture was cooled to room temperature, filtered through a pad of Celite®, and concentrated. Flash column chromatography (gradient, 35-70% EtOAc/hexanes) gave 2-(4-chloro-1H-pyrrolo [3,2-c]pyridin-1-yl)-N,N-diethylacetamide (12-1) as an off-white solid: ¹H-NMR (500 MHz, CDCl₃) δ 8.08 (d, J=5.61H, 1H), 7.17 (d, J=3.42 Hz, 1H), 7.09 (d, 3=5.86 Hz, 1H), 6.71 (d, J=3.17 Hz, 1H), 4.90 (s, 2H), 3.41 (m, 4H), 1.24 (t, J=7.08 Hz, 3H), 1.15 (t, J=7.08 Hz, 3H).

2-(4-amino-1H-pyrrolo[3,2-c]pyridin-1-yl)-N,N-diethylacetamide (12-2)

A solution of 2-(4-chloro-1H-pyrrolo[3,2-c]pyridin-1-yl)-N,N-diethylacetamide (12-1, 52 mg, 0.2 mmole), benzophenone imine (0.04 mL, 0.24 mmole), NaOtBu (26 mg, 0.27 mmole), Pd₂(dba)₃ (4 mg) and racemic BINAP (7 mg) in dry toluene (2 mL) was degassed then heated to 80° C. After 18 hours, the mixture as cooled and concentrated. The residue was taken up in MeOH (2 mL) and hydroxylamine (0.1 mL of a 50% solution in H₂O) was added. After 5 hours, the mixture was concentrated. Flash column chromatography (gradient, 0-10% MeOH/CH₂Cl₂ then 10% MeOH(CHCl₃ saturated with NH₃) gave 2-(4-amino-1H-pyrrolo[3,2-c]pyridin-1-yl)-N,N-diethylacetamide as a pale yellow solid: ¹H-NMR (500 MHz, CDCl₃) δ 7.77 (d, J=6.10 Hz, 1H), 7.02 (d, J=3.17 Hz, 1H), 6.63 (d, J=6.11 Hz, 1H), 6.48 (d, J=3.17 Hz, 1H), 4.84 (s, 2H), 3.40 (m, 4H), 1.67 (bs, 1H), 1.19 (m, 6H).

2-{4-[(5-cyano-1,3-thiazol-2-yl)amino]-1H-pyrrolo[3,2-c]pyridin-1-yl}-N,N-diethylacetamide (12-3)

To a solution of 2-(4-amino-1H-pyrrolo[3,2-c]pyridin-1-yl)-N,N-diethylacetamide (12-2, 21 mg, 0.085 mmole) in dry THF (1 mL) was added NaH (10 mg, 60% dispersion in mineral oil, 0.21 mmole). After gas evolution had ceased 2-chloro-5-cyanothiazole (18 mg, 0.13 mmole) was added and the mixture heated to reflux. After 4 hours, the mixture was cooled to room temperature, quenched with saturated NH₄Cl, and extracted with EtOAc (3×). The combined organic layers were dried (MgSO₄), filtered, and concentrated. The residue was triturated with Et₂O/hexanes to give the title compound as a yellow solid: ¹H-NMR (500 MHz, d₆-DMSO) δ 12.39 (s, 1H), 8.31 (s, 1H), 7.99 (d, J=5.62 Hz, 1H), 7.34 (s, 1H), 7.12 (s, 2H), 5.21 (s, 2H), 3.45 (m, 2H), 3.31 (m, 2H), 1.22 (t, J=6.83 Hz, 3H), 1.04 (t, J=7.08 Hz, 3H).

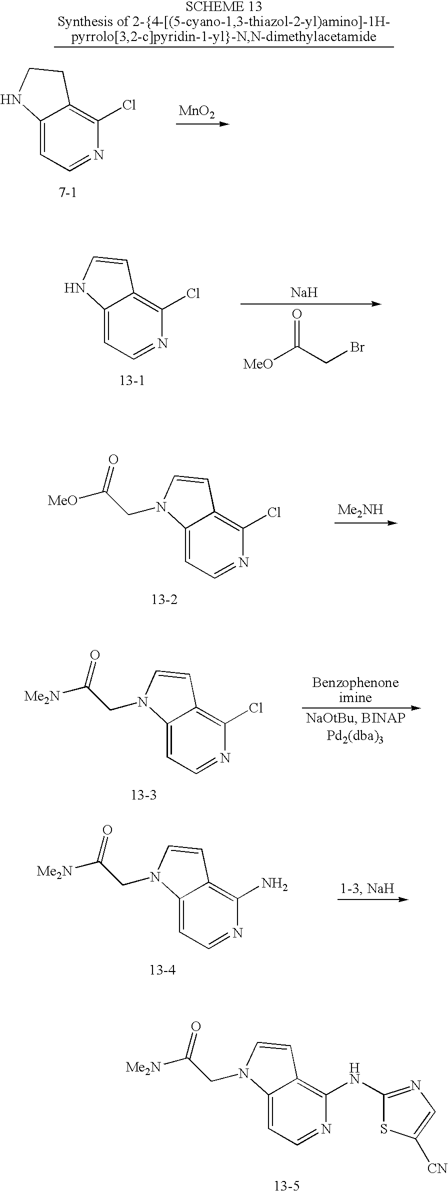

4-chloro-1H-pyrrolo[3,2-c]pyridine (13-1)

To a solution of 4-chloro-2,3-dihydro-1H-pyrrolo[3,2-c]pyridine (494 mg, 3.20 mmole) in dry THF (15 mL) was added MnO₂ (1.39 g, 15.98 mmole) and the mixture was heated to reflux. After 6 hr the mixture was cooled to RT, filtered through a pad of Celite®, and concentrated to give 4-chloro-1H-pyrrolo[3,2-c]pyridine as a white solid (490 mg, 100%): ¹H-NMR (500 MHz, CDCl₃) δ 8.79 (s, 1H), 8.09 (d, J=5.62 Hz, 1H), 7.28 (m, 2H), 6.71 (d, J=2.19 Hz, 1H).

methyl(4-chloro-1H-pyrrolo[3,2-c]pyridin-1-yl)acetate (13-2)

To a solution of 4-chloro-1H-pyrrolo[3,2-c]pyridine (200 mg, 1.31 mmole) in dry THF (7 mL) was added NaH (60 mg, 60% dispersion in mineral oil, 1.57 mmole) at 0° C. After gas evolution had ceased methyl bromoacetate (0.16 mL, 1.70 mmole) was added and the mixture warmed to RT. After 1 hr the mixture was diluted with saturated NH₄Cl and extracted with EtOAc (3×). The combined organic layers were dried (MgSO₄), filtered, and concentrated. The residue was triturated with Et₂O/hexanes to give methyl(4-chloro-1H-pyrrolo[3,2-c]pyridin-1-yl)acetate as an off-white solid (240 mg, 81%): ¹H-NMR (500 MHz, CDCl₃) δ 8.11 (d, J=5.86 Hz, 1H), 7.15 (d, J=3.18 Hz, 1H), 7.11 (d, J=5.86 Hz, 1H), 6.72 (d, J=3.41 Hz, 1H), 4.87 (s, 2H), 3.78 (s, 3H).

2-(4-chloro-1H-pyrrolo[3,2-c]pyridin-1-yl)-N,N-dimethylacetamide (13-3)

Methyl(4-chloro-1H-pyrrolo[3,2-c]pyridin-1-yl)acetate (120 mg, 0.53 mmole) was taken up in 3 mL 2M dimethylamine in MeOH. After 18 hr the mixture was concentrated. The residue was triturated with Et₂O and concentrated to give 2-(4-chloro-1H-pyrrolo[3,2-c]pyridin-1-yl)-N,N-dimethylacetamide as a pale yellow solid (138 mg, 100%): ¹H-NMR (500 MHz, CDCl₃) δ 8.08 (d, J=5.86 Hz, 1H), 7.14 (d, J=3.42 Hz, 1H), 7.11 (d, J=4.88 Hz, 1H), 6.71 (d, J=2.44 Hz, 1H), 4.92 (s, 2H), 3.11 (s, 3H), 3.01 (s, 3H).

2-(4-amino-1H-pyrrolo[3,2-c]pyridin-1-yl)-N,N-dimethylacetamide (13-4)

A solution of 2-(4-chloro-1H-pyrrolo[3,2-c]pyridin-1-yl)-N,N-dimethylacetamide (138 mg, 0.58 mmole), benzophenone imine (0.12 mL, 0.7 mmole), NaOtBu (78 mg, 0.81 mmole), Pd₂(dba)₃ (11 mg) and racemic BINAP (22 mg) in dry toluene (3 mL) was degassed then heated to 80° C. After 18 hr the mixture was cooled and concentrated. The residue was taken up in MeOH (3 mL) and hydroxylamine (0.1 mL of a 50% solution in H₂O) was added. After 3 hr the mixture was filtered through a pad of Celite® and concentrated. Flash column (gradient, 0-10% MeOH/CHCl₃ saturated with NH₃) gave 2-(4-amino-1H-pyrrolo[3,2-c]pyridin-1-yl)-N,N-dimethylacetamide as a pale yellow foam (65 mg, 51%): ¹H-NMR (500 MHz, CDCl₃) δ 7.80 (d, J=5.86 Hz, 1H), 6.99 (d, J=3.17 Hz, 1H), 6.64 (d, J=5.86 Hz, 1H), 6.47 (d, J=3.17 Hz, 1H), 4.85 (s, 2H), 4.72 (bs, 2H), 3.06 (s, 3H), 3.00 (s, 3H).

2-{4[(5-cyano-1,3-thiazol-2-yl)amino]-1H-pyrrolo[3,2-c]pyridin-1-yl}-N,N-dimethylacetamide (13-5)

To a solution of 2-(4-amino-1H-pyrrolo[3,2-c]pyridin-1-yl)-N,N-dimethylacetamide (65 mg, 0.3 mmole) in dry THF (2 mL) was added NaH (30 mg, 60% dispersion in mineral oil, 0.75 mmole). After gas evolution had ceased 2-chloro-5-cyanothiazole (65 mg, 0.45 mmole) was added and the mixture heated to reflux. After 4 hr a gum had formed. Additional NaH (30 mg) and 1 mL dry DMF was added and heating continued. After 3 hr the mixture was cooled to RT and quenched with saturated NH₄Cl. The resulting mixture was concentrated to dryness. Purification by reverse phase HPLC (5-100% CH₃CN/H₂O+0.1% TFA) gave mixed fractions. Fractions containing the product were pooled and concentrated. Flash column (10% MeOH(CH₂Cl₂) gave 2-{4-[(5-cyano-1,3-thiazol-2-yl)amino]-1H-pyrrolo[3,2-c]pyridin-1-yl}-N,N-dimethylacetamide as a yellow solid (27 mg, 28%) after trituration with Et₂O: ¹H-NMR (500 MHz, d⁶-DMSO) δ 12.37 (s, 1H), 8.30 (s, 1H), 7.98 (d, J=5.86 Hz, 1H), 7.29 (d, J=3.18 Hz, 1H), 7.17 (d, J=5.86 Hz, 1H), 7.12 (s, 1H), 5.21 (s, 2H), 3.10 (s, 3H), 2.86 (s, 3H); MS(ES) (M+H)⁺ 327.

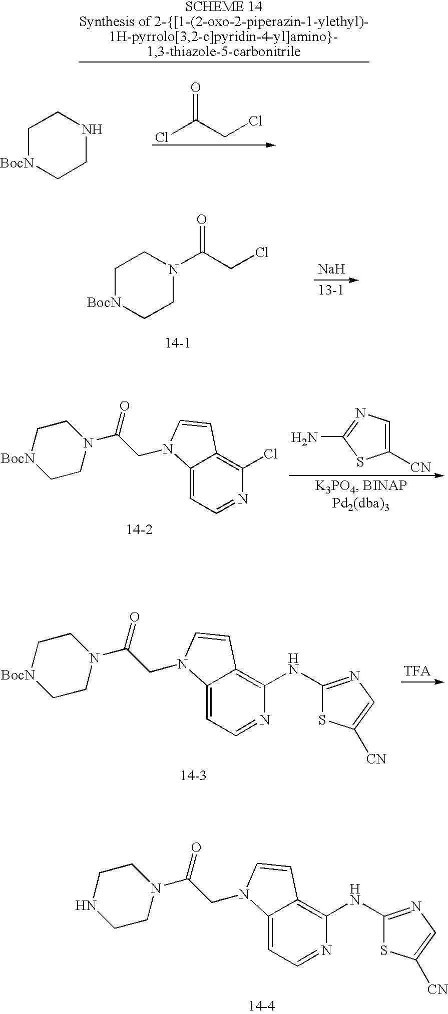

tert-butyl 4-(chloroacetyl)piperazine-1-carboxylate (14-1)

To a solution of tert-butyl piperazine-1-carboxylate (1.0 g, 5.37 mmole) in CH₂Cl₂ (25 mL) was added Et₃N (0.9 mL, 6.44 mmole) then chloroacetylchloride (0.47 mL, 5.91 mmole) slowly at 0° C. After 30 min the mixture was warmed to RT. After 2 hr the mixture was concentrated. The residue was taken up in EtOAc and filtered through a pad of silica gel (EtOAc as eluent). The filtrated was concentrated to give tert-butyl 4-(chloroacetyl)piperazine-1-carboxylate as a light amber oil (1.52 g, 100%): ¹H-NMR (500 M&, CDCl₃) δ 4.08 (s, 2H), 3.59 (d, J=4.88 Hz, 2H), 3.51 (s, 4H), 3.45 (d, J=5.13 Hz, 2H), 1.48 (s, 9H).

tert-butyl 4-[(4-chloro-1H-pyrrolo[3,2-c]pyridin-1-yl)acetyl]piperazine-1-carboxylate (14-2)

To a solution of 4-chloro-1H-pyrrolo[3,2-c]pyridine (13-1, 100 mg, 0.66 mmole) in dry THF (2 mL) was added NaH (32 mg, 60% dispersion in mineral oil, 0.79 mmole). After gas evolution had ceased tert-butyl 4-(chloroacetyl)piperazine-1-carboxylate (224 mg, 0.85 mmole) in dry THF (1 mL) was added. After 1 hr the mixture was quenched with saturated NH₄Cl and extracted with EtOAc (3×). The combined organic layers were dried (MgSO₄), filtered, and concentrated. Flash column (60% EtOAc/hexanes) gave tert-butyl 4-[(4-chloro-1H-pyrrolo[3,2-c]pyridin-1-yl)acetyl]piperazine-1-carboxylate as a white solid (195 mg, 79%): ¹H-NMR (500 MHz, CDCl₃) δ 8.10 (d, J=5.86 Hz, 1H), 7.14 (d, J=3.42 Hz, 1H), 7.12 (d, J=5.12 Hz, 1H), 6.73 (d, J=2.44 Hz, 1H), 4.95 (s, 2H), 3.62 (bs, 2H), 3.45 (bs, 6H), 1.48 (s, 9H).

tert-butyl 4-({4-[(5-cyano-1,3-thiazol-2-yl}amino]-1H-pyrrolo[3,2-c]pyridin-1-yl acetyl)piperazine-1-carboxylate (14-3)

A mixture of tert-butyl 4-[(4-chloro-1H-pyrrolo[3,2-c]pyridin-1-yl)acetyl]piperazine-1-carboxylate (50 mg, 0.13 mmole), 2-amino-5-cyanothiazole (20 mg, 0.16 mmole), K₃PO₄ (39 mg, 0.18 mmole), Pd₂(dba)₃ (2 mg), and xantphos (5 mg) in dry toluene (1 mL) was degassed then heated to reflux. After 1 hr the mixture was cooled to RT. Pd₂(dba)₃ (2 mg), xantphos (5 mg), and 1 mL dry dioxane were added and heating continued. After 18 hr the mixture was cooled, diluted with H₂O, and extracted with CH₂Cl₂ (3×). The combined organic layers were dried (MgSO₄), filtered, and concentrated. Flash column (gradient, 50-100% EtOAc/hexanes) gave tert-butyl 4-({4-[(5-cyano-1,3-thiazol-2-yl}amino]-1H-pyrrolo[3,2-c]pyridin-1-yl]acetyl)piperazine-1-carboxylate as a pale yellow solid (36 mg, 58%): MS(ES) (M+H)⁺ 468.

2-{[1-(2-oxo-2-piperazin-1-ylethyl)-1H-pyrrolo[3,2-c]pyridin-4-yl]amino}-1,3-thiazole-5-carbonitrile (14-4)

Tert-butyl 4-({4-[(5-cyano-1,3-thiazol-2-yl)amino]-1H-pyrrolo[3,2-c]pyridin-1-yl}acetyl)piperazine-1-carboxylate (36 mg, 0.08 mmole) was taken up in 2 mL CH₂Cl₂ and 2 mL TFA. After 1 hr the mixture was concentrated to dryness. Trituration with EtOAc gave the trifluoroacetate salt of 2-{[1-(2-oxo-2-piperazin-1-ylethyl)-1H-pyrrolo[3,2-c]pyridin-4-yl]amino}-1,3-thiazole-5-carbonitrile as an off-white solid (15 mg, 53%): ¹H-NMR (500 MHz, d⁶-DMSO) δ 12.42 (s, 1H), 8.80 (bs, 1H), 8.31 (s, 1H), 8.00 (d, J=5.86 Hz, 1H), 7.27 (d, J=3.42 Hz, 1H), 7.20 (d, J=6.11 Hz, 1H), 7.14 (d, J=2.93 Hz, 1H), 5.30 (s, 2H), 3.76 (bs, 2H), 3.65 (bs, 2H), 3.26 (bs, 2H), 3.12 (s, 2H); MS(ES) M+H)⁺ 368.

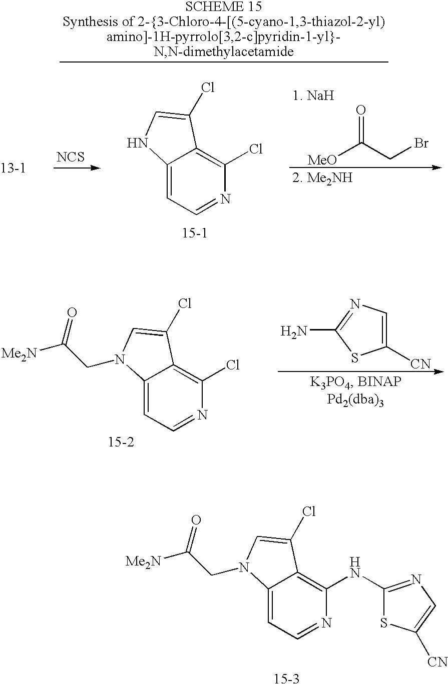

2-{3-Chloro-4-[(5-cyano-1,3-thiazol-2-yl)amino]-1H-pyrrolo[3,2-c]pyridin-1-yl}-N,N-dimethylacetamide (15-3)

4-Chloro-1H-pyrrolo[3,2-c]pyridine (100 mg, 0.66 mmole) and NCS (96 mg, 0.72 mmole) were combined in DCE (5 mL) at RT. After 18 hr the mixture was heated to reflux. After 2 hr the mixture was cooled to RT and concentrated. The residue was taken up in CH₂Cl₂ and filtered to give a tan solid. The filtrate was concentrated. Flash column (40% EtOAc/hexanes) of the filtrate gave a light tan solid. The solids were combined (86 mg, 70%) and used in the next step without further purification. ¹H NMR indicated a 3:1 ratio of 2-chloro (15-1) and 2,3-dichloro products.

To the above solid (86 mg, 0.46 mmole) in dry THF (3 mL) was added NaH (22 mg, 60% dispersion in mineral oil, 0.55 mmole) at RT. After gas evolution had ceased methyl bromoacetate (0.06 mL, 0.6 mmole) was added. After 1 hr the mixture was diluted with saturated NH₄Cl and extracted with EtOAc (3×). The combined organic layers were dried (MgSO₄), filtered, and concentrated. The residue was triturated with hexanes to give a light tan solid (89 mg, 75%) which was used in the next step without further purification.

The above solid (89 mg, 0.34 mmole) was taken up in 2M dimethylamine in THF (3 mL) at RT. After 5 days the mixture was concentrated. Flash column (100% EtOAc) gave a clear glass (15-2, 68 mg, 73%) which was used in the next step without further purification.

The above solid (68 mg, 0.25 mmole), 2-amino-5-cyanothiazole (38 mg, 0.30 mmole), K₃PO₄ (74 mg, 0.35 mmole), Pd₂(dba)₃ (5 mg), and xantphos (9 mg) were combined in dry dioxane (2 mL). The mixture was degassed then heated to reflux. After 18 hr additional Pd₂(dba)₃ (5 mg) and xantphos (9 mg) were added and heating continued. After 18 hr the mixture was cooled to RT, diluted with H₂O, and extracted with CH₂Cl₂ (3×). The combined organic layers were dried (MgSO₄), filtered, and concentrated. Purification by reverse phase HPLC (5-100% CH₃CN/H₂O+0.1% TFA) gave 2-{3-chloro-4-[(5-cyano-1,3-thiazol-2-yl)amino]-1H-pyrrolo[3,2-c]pyridin-1-yl}-N,N-dimethylacetamide with 2-{2,3-dichloro-4 [(5-cyano-1,3-thiazol-2-yl)amino]-1H-pyrrolo[3,2-c]pyridin-1-yl}-N,N-dimethylacetamide (15-3, 8:1 ratio as estimated by ¹H-NMR) as a yellow solid (16 mg, 18%): major: ¹H-NMR (500 MHz, d⁶-DMSO) δ 10.20 (s, 1H), 8.34 (s, 1H), 8.05 (d, J=6.15 Hz, 1H), 7.55 (s, 1H), 7.29 (d, J=5.86 Hz, 1H), 5.22 (s, 2H), 3.30 (s, 3H), 3.09 (s, 3); minor: 1H-NMR (500, d⁶-DMSO) δ 8.20, 7.70, 7.40, 7.05, 5.19.

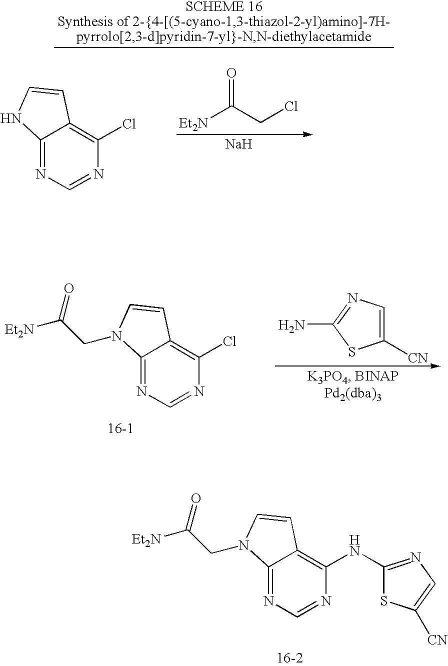

2-(4-chloro-7H-pyrrolo[2,3-d]pyrimidin-7-yl)-N,N-diethylacetamide (16-1)

4-Chloro-7H-pyrrolo[2,3-d]pyrimidine (J. Davoll J. Chem. Soc. 1960 131-138., 0.093 g, 0.61 mmol) was dissolved in anhydrous DMF (2 mL) in a flame dried round bottom flask. The reaction was cooled to 0° C. Sodium hydride (60% dispersion in mineral oil) (0.035 g, 1.46 mmol) and 2-chloro-N,N-diethyl acetamide (91.8 uL, 0.67 mmol) were added. The reaction was evacuated and filled with N₂ (3×) and was allowed to warm to rt. After 3 h, water was added and the pdt was extracted with EtOAc (4×). The combined organic layers were dried (Na₂SO₄), filtered, and concentrated to afford a light brown oil. The oil was purified by reverse phase chromatography (gradient, 5-100% CH₃CN/H₂O+0.1% TFA). The fractions containing the desired compound were concentrated to dryness to afford the product as an off white solid. Free base: ¹H NMR (DMSO-d₆) δ 8.61 (s, 1H), 7.69 (d, 1H, J=3.67 Hz), 6.65 (d, 1H, J=3.66 Hz), 5.25 (s, 2H), 3.46 (q, 2H, J=7.08 Hz), 3.28 (q, 2H, J=7.08 Hz), 1.25 (t, 3H, J=7.08 Hz), 1.03 (t, 3H, J=7.08 Hz).

2-{4-[(5-cyano-1,3-thiazol-2-yl)amino]-7H-pyrrolo[2,3-d]pyrimidin-7-yl}-N,N-diethylacetamide (16-2)

2-Amino-1,3-thiazole-5-carbonitrile (0.033 g, 0.26 mmol), xantphos (0.008 g, 0.01 mmol), Pd₂(dba)₃ (0.004 g, 0.01 mmol), and K3PO4 (0.069 g, 0.72 mmol) were combined in a flame dried round bottom flask. The reaction was evacuated and filled with N₂ (3×). A suspension of 2-(4-chloro-7H-pyrrolo[2,3-d]pyrimidin-7-yl)-N,N-diethylacetamide (0.058 g, 0.22 mmol) in anhydrous toluene (2 mL) was added. The reaction was evacuated and filled with N₂ (3×) and heated to 120° C. After 3 h, more xantphos (0.008 g, 0.01 mmol), Pd₂(dba)₃ (0.004 g, 0.01 mmol) were added and the reaction was heated to 120° C. for 20 h. The reaction was cooled to rt and concentrated in vacuo to afford a dark brown solid. The solid was dissolved in MeOH and filtered through celite. The filtrate was concentrated in vacuo to afford an orange solid. The solid was purified by reverse phase chromatography (gradient, 5-100% CH₃CN/H₂O+0.1% TFA). The fractions containing the desired compound were concentrated to dryness to afford the product as white needle-like crystals. Free base: ¹H NMR (DMSO-d₆) δ 12.86 (bs, 1H), 8.58 (s, 1H), 8.38 (s, 1H), 7.43 (d, 1H, J=3.41 Hz), 7.02 (d, 1H, J=3.42 Hz), 5.19 (s, 2H), 3.47 (q, 2H, J=7.32 Hz), 3.28 (m, 2H), 1.25 (t, 3H, J=7.08 Hz), 1.03 (t, 3H, J=7.08 Hz). [M+H]+=356.1270.

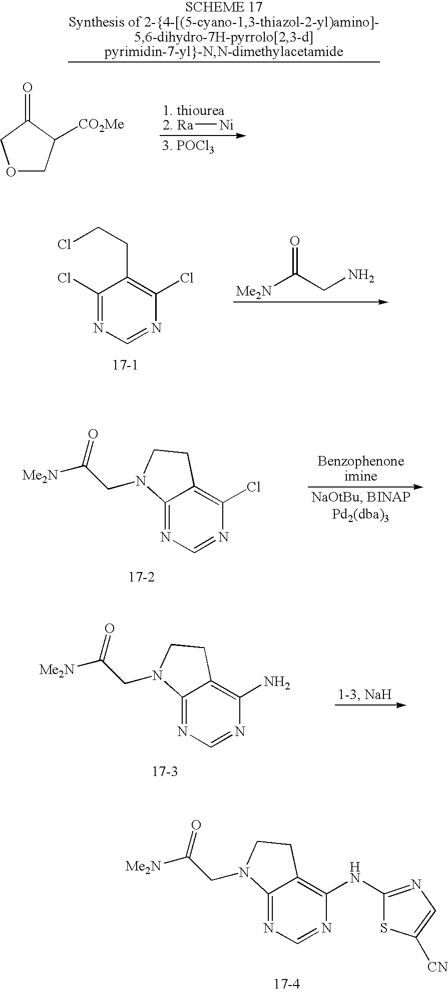

4,6-dichloro-5-(2-chloroethyl)pyrimidine (17-1)

An oven-dried flask under N₂ was charged with 20 ml of absolute EtOH and sodium (1.00 g, 41.6 mmol) was added in small chunks until bubbling had subsided. The resulting solution was warmed to 40° C. and thiourea (2.38 g, 31.2 mmol) was added. After 10 min methyl 2-oxotetrahydrofuran-3-carboxylate (Syn Comm 1989, 19, 1389-1393, 3.00 g, 20.8 mmol) was added dropwise over 30 min. The reaction was stirred at 40° C. for 3 h during which time a white precipitate forms. The reaction was cooled to ambient temperature and was concentrated in vacuo foolowed by addition of 50 ml water and adjustment of the pH to 1 with concentrated aqueous HCl. Very little precipitate formed so the mixture was concentrated in vacuo. A ¹H NMR of the residue showed primarily 5-(2-hydroxyethyl)-2-mercaptopyrimidine-4,6-diol so it was used directly in the next transformation.

To a flask containing 5-(2-hydroxyethyl)-2-mercaptopyrimidine-4,6-diol was added 22 g of a 50% Ra—Ni aqueous slurry. The reaction was diluted to 50 ml with water and 1 ml of concentrated aqueous NH₃ was added. After 1 h an additional 5 g of the Ra—Ni slurry was added and the reaction was stirred for 30 h. The reaction was filtered through celite, and washed with a minimum of water. The filtrate was concentrated in vacuo then redissolved in 10 ml water and refridgerated. The resulting precipitate was filtered and washed with cold water. The filtrate was concentrated in vacuo.

The filtered solid and concentrated filtrate were treated in three separate batches with POCl₃ (as solvent) at reflux for 18 h. The reactions were concentrated to dryness, quenched with ice and neutralized with NaHCO₃ (s). The resulting mixtures were extracted 3× with DCM, and the organic layers were dried over Na₂SO₄, filtered and concentrated to afford a total of 492 mg of 4,6-dichloro-5-(2-chloroethyl)pyrimidine. ¹H NMR (CDCl₃) δ 8.70 (s, 1H), 3.79 (t, 2H, J=7.3 Hz), 3.42 (t, 2H, J=7.3 Hz).

2-(4-chloro-5,6-dihydro-7H-pyrrolo[2,3-d]pyrimidin-7-yl)-N,N-dimethylacetamide (17-2)

An oven-dried flask under N2 was charged with NaH (60% dispersion in mineral oil, 45 mg, 1.13 mmol) and N,N-dimethylglycinamide acetate (Bachem, 184 mg, 1.14 mmol) and anhydrous THF, 3 ml was added. Once the resultig bubbling had subsided 4,6-dichloro-5-(2-chloroethyl)pyrimidine (200 mg, 0.946 mmol) was added and the reaction was heated to 60° C. After 2 h LCMS showed complete conversion to the product of displacement of a chloropyrimidine function by the amine. Additional NaH (45 mg, 1.13 mmol) was added and the reaction was continued at 60° C. After 3 h additional NaH (25 mg, 0.63 mmol) was added. After 4 h at 60° C. the reaction was cooled to ambient temperature and was diluted with half-saturated aqueous NaHCO₃. The mixture was extracted 3× with DCM and the organic phases were dried over Na₂SO₄, filtered and concentrated in vacuo. Purification by flash column chromatography (elution gradient: DCM to 95:5 DCM/MeOH) afforded 181 mg as an off-white solid. ¹H NMR (CDCl₃) δ 8.18 (s, 1H), 4.23 (s, 2H), 3.79 (t, 211, J=8.6 Hz), 3.12 (t, 2H, J=8.7 Hz) 3.05 (s, 31, 2.97 (s, 3H).

2-(4-amino-5,6-dihydro-7H-pyrrolo[2,3-d]pyrimidin-7-yl)-N,N-dimethylacetamide (17-3)

2-(4-Chloro-5,6-dihydro-7H-pyrrolo[2,3-d]pyrimidin-7-yl)-N,N-dimethylacetamide (0.073 g, 0.30 mmol), racemic BINAP (0.011 g, 0.02 mmol), Pd₂(dba)₃ (0.006 g, 0.01 mmol), and sodium tert-butoxide (0.041 g, 0.43 mmol) were combined in a flame dried round bottom flask. Anhydrous toluene (1.5 mL) was added and the reaction was evacuated and filled with N₂ (3×). Benzophenone imine (61.2 uL, 0.36 mmol) was added and the reaction was heated to 80° C. After 3 h, the reaction was cooled to rt and concentrated in vacuo to afford a yellow/brown oil. The oil was dissolved in MeOH (2 mL) and treated with hydroxylamine (50% aq. solution, 27.9 uL, 0.91 mmol). The reaction was stirred at rt for 15.5 h. More hydroxylamine (50% aq. solution, 27.9 uL, 0.91 mmol) was added. After 5.5 h, the reaction was filtered through celite. The filtrate was concentrated in vacuo to afford a yellow/green solid. The solid was purified by flash column chromatography (100% CHCl₃ saturated with NH₃ to 90:10 CHCl₃ saturated with NH₃:MeOH). The fractions containing the desired compound were concentrated to dryness to afford the free base as a white solid. Free base: ¹H NMR (CD₃OD) δ 8.03 (s, 1H), 4.30 (s, 2H), 3.69 (m, 2H, overlapping with impurity), 3.08 (s, 3H), 2.99 (t, 2H, J=9.04 Hz), 2.94 (s, 3H).

2-{4-[(5-cyano-1,3-thiazol-2-yl)amino]-5,6-dihydro-7H-pyrrolo[2,3-d]pyrimidin-7-yl}-N,N-dimethylacetamide (17-4)

2-(4-Amino-5,6-dihydro-7H-pyrrolo[2,3-d]pyrimidin-7-yl)-N,N-dimethylacetamide (0.025 g, 0.11 mmol) was dissolved in TBF (1 mL). Sodium hydride (60% dispersion in mineral oil) (0.011 g, 0.45 mmol) and 2-chloro-1,3-thiazole-5-carbonitrile (0.020 kg, 0.14 mmol) were added and the reaction was heated to 75° C. After 6.5 h, more sodium hydride (0.011 g, 0.45 mmol) and 2-chloro-1,3-thiazole-5-carbonitrile (0.020 g, 0.14 mmol) were added. The reaction was heated to 75° C. for 16 h. The reaction was allowed to cool to rt. H₂O was added and 1N HCl was used to adjust to pH 6. The resulting yellow precipitate was collected by vacuum filtration and washed with water. The filtrate was then extracted with CH₂Cl₂ (4×). The combined organic layers were dried (Na₂SO₄), filtered, and concentrated to afford a yellow oil. The oil and the precipitate were combined and purified by reverse phase chromatography (gradient, 5-100% CH₃CN/H₂O+0.1% TFA). The fractions containing the desired compound were concentrated to dryness to afford the free base as a colorless oil. The oil was azeotroped with MeOH (2×) and EtOAc/hexanes to afford the product as a white solid. Free base: ¹H NMR (CD₃OD) δ 8.30 (s, 1H), 8.04 (s, 1H), 4.38 (s, 2H), 3.81 (t, 2H, J=8.79 Hz), 3.14 (t, 2H, J=8.79 Hz), 3.08 (s, 3H), 2.96 (s, 3H). [M+H]+=330.1131.

1. A compound of Formula I 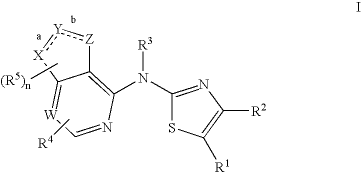
or a pharmaceutically acceptable salt or stereoisomer thereof, wherein
a and b are a single bond or a double bond provided both a and b are not a double bond at the same time;
X, Y and Z are C, S, N or O provided that at least one of X, Y and Z is C;
W is C or N;
n is 0 through 6;
R¹ is:
1) H.
2) Oᵣ(C₁-C₆)perfluoroalkyl,
3) OH,
4) CN,
5) halogen,
6) (C═O)ᵣOₛ(C₁-C₁₀)alkyl,
7) (C═O)ᵣOₛ(C₂-C₁₀)alkenyl,
8) (C═O)ᵣOₛ(C₂-C₁₀)alkynyl,
9) (C═O)ᵣOₛaryl,
10) (C═O)ᵣOₛheterocyclyl, or
11) (C₀-C₆)alkyl-NR^{a}R^{b},
wherein r and s are independently 0 or 1, and said alkyl, alkenyl, alkynyl, aryl and heterocyclyl is optionally substituted with one or more substituents selected from R⁵;
R² is:
1) H,
2) Oᵣ(C₁-C₆)perfluoroalkyl,
3) OH,
4) CN,
5) halogen,
6) (C═O)ᵣOₛ(C₁-C₁₀)alkyl,
7) (C═O)ᵣOₛ(C₂-C₁₀)alkenyl,
8) (C═O)ᵣOₛ(C₂-C₁₀)alkynyl,
9) (C═O)ᵣOₛaryl,
10) (C═O)ᵣOₛheterocyclyl, or
11) (C₀-C₆)alkyl-NR^{a}R^{b},
wherein r and s are independently 0 or 1, and said alkyl, alkenyl, alkynyl, aryl and heterocyclyl is optionally substituted with one or more substituents selected from R⁵;
R³ is:
1) H,
2) SO₂R^{c},
3) (C═O)ᵣR^{c}, wherein r is 0 or 1, or
4) CO₂R^{c};
R⁴ is:
1) H,
2) Oᵣ(C₁-C₆)perfluoroalkyl,
3) OH,
4) CN,
5) halogen,
6) (C═O)ᵣOₛ(C₁-C₁₀)alkyl,
7) (C═O)ᵣOₛ(C₂-C₁₀)alkenyl,
8) (C═O)ᵣOₛ(C₂-C₁₀)alkynyl,
9) (C═O)ᵣOₛaryl,
10) (C═O)ᵣOₛheterocyclyl, or
11) (C₀-C₆)alkyl-NR^{a}R^{b},
wherein r and s are independently 0 or 1, and said alkyl, alkenyl, alkynyl, aryl and heterocyclyl is optionally substituted with one or more substituents selected from R⁵;
R⁵ is:
1) H,
2) SO₂R^{c},
3) (C═O)ᵣR^{c},
4) CO₂R^{c},
5) Oᵣ(C═O)ₛNR^{a}R^{b},
6) (C═O)ᵣOₛaryl,
7) (C═O)ᵣOₛ-heterocyclyl,
8) halogen,
9) OH,
10) oxo,
11) O(C₁-C₃)perfluoroalkyl,
12) (C₁-C₃)perfluoroalkyl,
13) (C═O)ᵣOₛ(C₁-C₁₀)alkyl,
14) CHO,
15) CO₂H, or
16) CN,
wherein r and s are independently 0 or 1, and said alkyl, aryl, and heterocyclyl are optionally substituted with one or more substituents selected from R^{d};
R^{a} and R^{b} are independently:
1) H,
2) (C═O)ᵣ(C₁-C₁₀)alkyl,
3) S(O)₂R^{c},
4) (C═O)ᵣheterocyclyl,
5) (C═O)ᵣaryl, or
6) CO₂R^{c},
wherein r is 0 or 1 and said allyl, heterocyclyl, and aryl optionally substituted with one or more substituents selected from R^{d}, or
R^{a} and R^{b} are taken together with the nitrogen to which they are attached to form a monocyclic or bicyclic heterocycle with 5-7 members in each ring and optionally containing, in addition to the nitrogen, one or two additional heteroatoms selected from N, O and S, said monocyclic or bicyclic heterocycle optionally substituted with one or more substituents selected from R^{d};
R^{c} is
(C₁-C₆)alkyl, aryl, benzyl, or heterocyclyl;
R^{d} is
1) (C═O)ᵣOₛ(C₁-C₁₀)alkyl, wherein r and s are independently 0 or 1, optionally substituted with up to three substituents selected from OH, (C₁-C₆)alkoxy, halogen, CN, oxo, N(R^{e})₂ and S(O)₂R^{c},
2) (C═O)N(R^{e})₂,
3) Oᵣ(C₁-C₃)perfluoroalkyl,
4) (C₀-C₆)alkylene-S(O)ₘR^{c}, wherein m is 0, 1 or 2,
5) oxo,
6) OH,
7) halogen,
8) CN,
9) (C₀-C₆)alkylene-aryl, optionally substituted with up to three substituents selected from R^{e},
10) (C₀-C₆)alkylene-heterocyclyl, optionally substituted with up to three substituents selected from R^{e},
11) (C₀-C₆)alkylene-N(R^{e})₂,
12) C(O)R^{c},
13) CO₂R^{c},
14) C(O)H, or
15) CO₂H; and
R^{e} is
H, (C₁-C₆)alkyl, aryl, heterocyclyl or S(O)R^{c}.

2. The compound of claim 1, wherein W is C or N; R¹ is CN or phenyl; and R², R³ and R⁴ are H.

3. The compound of claim 2, wherein W is C and R¹ is CN.

4. A compound selected from:
2-(2,3-dihydrofuro[2,3-c]pyridin-7-ylamino)-1,3-thiazole-5-carbonitrile;
2-{[3-(hydroxymethyl)-2,3-dihydrofuro[2,3-c]pyridin-7-yl]amino}-1,3-thiazole-5-carbonitrile;
2-[(1-methyl-1H-pyrazolo[4,3-c]pyridin-4-yl)amino]-1,3-thiazole-5-carbonitrile;
2-(2,3-dihydro-1H-pyrrolo[3,2-c]pyridin-4-ylamino)-1,3-thiazole-5-carbonitrile;
2-(1H-pyrrolo[3,2-c]pyridin-4-ylamino)-1,3-thiazole-5-carbonitrile;
2-{[1-(methylsulfonyl)-2,3-dihydro-1H-pyrrolo[3,2-c]pyridin-4-yl]amino}-1,3-thiazole-5-carbonitrile;
4-[(5-cyano-1,3-thiazol-2-yl)amino]-N,N-dimethyl-2,3-dihydro-1H-pyrrolo[3,2-c]pyridine-1-carboxamide;
2-[(1-methyl-2-oxo-2,3-dihydro-1H-imidazo[4,5-c]pyridin-4-yl)amino]-1,3-thiazole-5-carbonitrile;
2-(thieno[3,2-c]pyridin-4-ylamino)-1,3-thiazole-5-carbonitrile;
2-(furo[3,2-c]pyridin-4-ylamino)-1,3-thiazole-5-carbonitrile;
2-(thieno[2,3-d]pyrimidin-4-ylamino)-1,3-thiazole-5-carbonitrile;
2-{4-[(5-cyano-1,3-thiazol-2-yl)amino]-1H-pyrrolo[3,2-c]pyridin-1-yl}-N,N-diethylacetamide;
2-{4-[(5-Cyano-1,3-thiazol-2-yl)amino]-1H-pyrrolo[3,2-c]pyridin-1-yl}-N,N-dimethylacetamide;
2-{[1-(2-oxo-2-piperazin-1-ylethyl)-1H-pyrrolo[3,2-c]pyridin-4-yl]amino}-1,3-thiazole-5-carbonitrile;
2-{3-Chloro-4-[(5-cyano-1,3-thiazol-2-yl)amino]-1H-pyrrolo[3,2-c]pyridin-1-yl}-N,N-dimethylacetamide;
2-{4-[(5-cyano-1,3-thiazol-2-yl)amino]-7H-pyrrolo[2,3-d]pyrimidin-7-yl}-N,N-diethylacetamide; and
2-{4-[(5-cyano-1,3-thiazol-2-yl)amino]-5,6-dihydro-7H-pyrrolo[2,3-d]pyrimidin-7-yl}-N,N-dimethylacetamide;
or a pharmaceutically acceptable salt or stereoisomer thereof.

5. The compound according to claim 1 which is 2-{4-[(5-cyano-1,3-thiazole-2-yl)amino]-1H-pyrrolo[3,2-c]pyridin-1-yl}-N,N-diethylacetamide 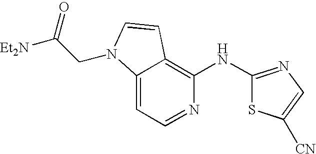
or a pharmaceutically acceptable salt thereof.

6. The compound according to claim 1 which is 2-(1H-pyrrolo[3,2-c]pyridin-4-ylamino)-1,3-thiazole-5-carbonitrile 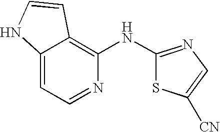
or a pharmaceutically acceptable salt thereof.

7. The compound according to claim 1 which is 2-{[1-(methylsulfonyl)-2,3-dihydro-1H-pyrrolo[3,2-c]pyridin-4-yl]amino}-1,3-thiazole-5-carbonitrile 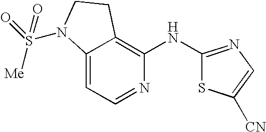
or a pharmaceutically acceptable salt thereof.

8. The compound according to claim 1 which is 4-[(5-cyano-1,3-thiazol-2-yl)amino]-N,N-dimethyl-2,3-dihydro-1H-pyrrolo[3,2-c]pyridine-1-carboxamide 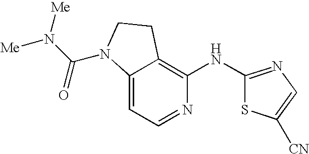
or a pharmaceutically acceptable salt thereof.

9. The compound according to claim 1 which is 2-{4-[(5-Cyano-1,3-thiazol-2-yl)amino]-1H-pyrrolo[3,2-c]pyridin-1-yl}-N,N-dimethylacetamide 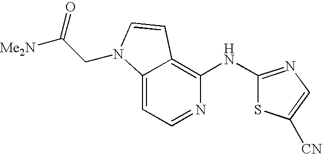
or a pharmaceutically acceptable salt thereof.

10. The compound according to claim 1 which is 2-{[1-(2-oxo-2-piperazin-1-ylethyl)-1H-pyrrolo[3,2-c]pyridin-4-yl]amino}-1,3-thiazole-5-carbonitrile 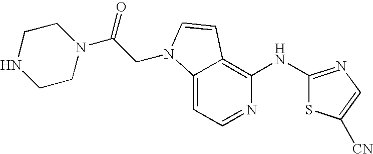
or a pharmaceutically acceptable salt thereof.

11. A pharmaceutical composition which is comprised of a compound in accordance with claim 1 and a pharmaceutically acceptable carrier.

12. A method of treating or preventing cancer in a mammal in need of such treatment which is comprised of administering to said mammal a therapeutically effective amount of a compound of claim 1.

13. A method of treating cancer or preventing cancer in accordance with claim 10 wherein the cancer is selected from cancers of the brain, genitourinary tract, lymphatic system, stomach, larynx and lung.

14. A method of treating or preventing cancer in accordance with claim 10 wherein the cancer is selected from histiocytic lymphoma, lung adenocarcinoma, small cell lung cancers, pancreatic cancer, glioblastomas and breast carcinoma.

15. A method of treating or preventing a disease in which angiogenesis is implicated, which is comprised of administering to a mammal in need of such treatment a therapeutically effective amount of a compound of claim 1.

16. A method in accordance with claim 13 wherein the disease is an ocular disease.

17. A method of treating or preventing retinal vascularization which is comprised of administering to a mammal in need of such treatment a therapeutically effective amount of compound of claim 1.

18. A method of treating or preventing diabetic retinopathy which is comprised of administering to a mammal in need of such treatment a therapeutically effective amount of compound of claim 1.

19. A method of treating or preventing age-related macular degeneration which is comprised of administering to a mammal in need of such treatment a therapeutically effective amount of a compound of claim 1.

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. A method of treating cancer which comprises administering a therapeutically effective amount of a compound of claim 1 in combination with radiation therapy and a compound selected from:
1) an estrogen receptor modulator,
2) an androgen receptor modulator,
3) retinoid receptor modulator,
4) a cytotoxic agent,
5) an antiproliferative agent,
6) a prenyl-protein transferase inhibitor,
7) an HMG-CoA reductase inhibitor,
8) an HIV protease inhibitor,
9) a reverse transcriptase inhibitor, and
10) another angiogenesis inhibitor.

32. (canceled)

33. (canceled)

34. (canceled)

35. (canceled)

36. (canceled)

37. (canceled)

38. (canceled)
